(19) United States
(12) Patent Application Publication
(10) Pub. No.: US 2022/0218814 A1
(43) Pub. Date: Jul. 14, 2022

(54) A VACCINE COMPRISING A NANOPARTICLE ENCAPSULATING EPITOPES AND ADJUVANT FOR NEUTRALIZING VIRUS INFECTION

(71) Applicants: ACADEMIA SINICA, Taipei (TW); NATIONAL TAIWAN UNIVERSITY, Taipei (TW)

(72) Inventors: Che-Ming Jack HU, Taipei City (TW); Hung-Chih YANG, Taipei City (TW)

(73) Assignees: ACADEMIA SINICA, Taipei (TW); NATIONAL TAIWAN UNIVERSITY, Taipei (TW)

(21) Appl. No.: 17/609,822

(22) PCT Filed: May 8, 2020
(86) PCT No.: PCT/US2020/032044

§ 371 Date: Nov. 9, 2021

Related U.S. Application Data
(60) Provisional application No. 62/846,035, filed on May 10, 2019.

(51) Int. Cl. A61K 39/12; A61K 39/39; A61K 9/51

(52) U.S. Cl. CPC A61K 39/12; A61K 2039/55555; A61K 9/5153; A61K 39/39

(57) ABSTRACT

We utilized a biocompatible hollow polymeric nanoparticle that coencapsulates T cell epitope peptides and oligodeoxynucleotide (ODN) CpG, and designed immunization strategies to evaluate its protectivity against influenza viruses in mice. This nanoparticle-based peptide vaccine adjuvanted with CpG stimulated robust antigen-specific CD4 and CD5 T cell immunity, but only caused minimal adverse effects compared with crude mixture of peptides and CpG. We used two peptides derived from the nucleocapsid protein (NP), MHC class I-restricted NP366-374 and MHC class ll-restricted NP311-325. This novel nanoparticle vaccine with two epitope peptides plus CpG induced robust and fully protective T cell immunity against influenza viruses. We demonstrates the utility of this novel hollow nanoparticle with co-encapsulation of only a pair of CD4+ and CD8+ T cell-stimulating influenza viral peptides and CpG in establishing near-sterilizing protective resident T cell immunity against heterosubtypic IAV infections, a critical step towards the development of universal influenza T cell vaccines.

The present application claims priority to U.S. Provisional Application 62/846,035, filed on May 10, 2019, and titled “Nanoparticles co-encapsulating peptides and CpG induce robust resident memory T cells confer ring near-sterilizing heterosubtypic immunity against lethal influenza virus infection,” the content of which is incorporated herein by reference in its entirety.

### FIELD

The present application relates to a vaccine and a method for manufacturing the same, and more particularly, to a vaccine comprising a nanoparticle encapsulating epitopes and adjuvant and a method for inducing robust resident memory T cells conferring near-sterilizing heterosubtypic immunity against lethal influenza virus infection.

### BACKGROUND

Influenza vaccine remains the most effective strategy to combat the threat of seasonal and pandemic influenza virus infections. Although effective, current inactivated influenza vaccines are succumbed to the frequently mutated viral surface proteins, namely, hemagglutinin (HA) and neuraminidase (NA), and fail to protect against distantly related strains or different subtypes. Thus, annual reformulation of influenza vaccines is often required to keep pace with ongoing viral evolution(1). In contrast, T cell immunity that recognizes conserved epitopes derived from the internal proteins of influenza A virus (IAV) likely provides cross protection against a broad spectrum of strains(2, 3). In animal studies, cross-reactive T cell immunity has been proved to provide heterosubtypic protection(4). Prior human studies have also demonstrated the preexisting cross-reactive T cell immunity against the emerging novel influenza viral strains and its association with favorable clinical outcomes(5-7). A very recent elegant application further discovered the T cell epitope peptides that are highly conserved across influenza A, B, and C viruses, justifying the development of T cell-based universal influenza vaccines(8).

Peptide-based T cell vaccines have attracted wide interest because they can stimulate desired epitope-specific T cell immunity against particular antigens(9, 10). However, peptides alone are usually not immunogenic, and tend to cause immunological tolerance(11). Overcoming the shortcomings of peptide vaccines is important for development of peptide-based T cell vaccines. Different strategies are utilized to enhance the immunogenicity of peptide-based T cell vaccines, including the use of viral and non-viral vaccine carriers(12). Viral vaccine carriers mimic natural viral infections and stimulate robust innate and adaptive immune responses, but raise potential biosafety concerns, whereas non-viral vectors are non-proliferating and avoid the safety risk, but usually have unsatisfying immunogenicity.

Nanoparticles are well suited for non-viral vaccine carrier application because they can be redirected for efficient uptake by professional antigen presenting cells (APCs), including dendritic cells (DCs) and macrophages(13, 14). Among different nanoparticle formulations, poly(D,L-lactide-co-glycolide) (PLGA) nanoparticles are an attractive vaccine platform due to their biodegradable nature and safety profiles(15, 16). Several properties of PLGA nanoparticle vaccines affect the ability to stimulate T cell immunity, including the size, encapsulation ability and the ability of in vivo uptake by APCs. Nanoparticles with co-encapsulation of antigenic peptides and CpG is advantageous for co-delivery into DCs to stimulate robust antigen-specific T cell immunity and prevents the systemic diffusion of small-molecule adjuvants that often causes systemic inflammatory reactions.

Although several vaccination strategies can induce robust systemic T cell immunity, they usually fail to prevent influenza virus infection. Recently, resident memory T cells (Trm) in lungs have been recognized as the first-line defense of T cell immunity against influenza virus infections(17, 18). Prior studies demonstrated Trm cells could provide near-sterilizing immunity to prevent the invading pathogens(19). Likewise, Trm cells induced by influenza virus infection in lungs play a critical role in controlling the influenza virus replications.

In this application, we utilized a novel biocompatible hollow PLGA nanoparticle that co-encapsulates antigenic peptides and CpG and designed appropriate peripheral subcutaneous priming and local lung boosting immunization strategy. With CpG plus mere two MHC class I-restricted and MHC class II-restricted peptides, this nanoparticle vaccine was able to stimulate both robust Trm cells in lungs and circulatory effector memory T cells (Tem) in mice. Of great interest, mice that were immunized with the nanoparticle vaccine co-encapsulating CpG and peptides by peripheral priming and local boosting were fully resistant to lethal infections of IAVs of different strains and subtypes. Given that highly conserved T cell epitope peptides were identified across influenza A, B and C viruses, our findings pave the way for developing universal influenza peptide-based T-cell vaccines.

### SUMMARY

One aspect of this invention is a vaccine, comprising:

a polymeric hollow nanoparticle encapsulating

one or more MHC class I epitopes;

one or more MHC class II epitopes; and

an adjuvant.

In one example, wherein the polymeric hollow nanoparticle has a diameter of 50-200 nm.

In one example, wherein the polymeric hollow nanoparticle is substantially composed of poly(D,L-lactide-co-glycolide) (PLGA).

In one example, wherein a lactide/glycolide ratio of the PLGA is about 40-60:60-40.

In one example, wherein an intrinsic viscosity of the PLGA is about 0.15-0.25 d L/g.

In one example, wherein the one or more MHC class I epitopes and the one or more MHC class II epitopes are independently antigenic peptides derived from a nucleocapsid protein of an influenza virus.

In one example, wherein the one or more MHC class I epitopes are nucleocapsid protein₃₆₆₋₃₇₄ consisting of the amino acid sequence of SEQ ID NO: 1, and the one or more MHC class II epitopes are nucleocapsid protein₃₁₁₋₃₂₅ consisting of the amino acid sequence of SEQ ID NO: 2.

In one example, wherein the adjuvant comprises MPLA, CpG-ODN, poly(I:C), or variants of cyclic-dinucleotides.

Another aspect of this invention is a method of manufacturing a vaccine, said vaccine comprising a polymeric hollow nanoparticle encapsulating one or more MHC class I epitopes, one or more MHC class II epitopes, and an adjuvant, comprising:

emulsifying a first solution comprising one or more MHC class I epitopes, one or more MHC class II epitopes and an adjuvant in a solvent comprising poly(D,L-lactide-co-glycolide) (PLGA);

sonicating the emulsion; and

purifying the polymeric hollow nanoparticle in the emulsion.

In one example, the method is further comprising:

adding a second solution to the emulsion after the sonicating step;

pouring the emulsion to water after the adding step; and

evaporating the solvent from the emulsion.

In one example, wherein the first solution comprises sodium bicarbonate.

In one example, wherein the concentration of the sodium bicarbonate ranges from 100-300 mM.

In one example, wherein the solvent comprises dichloromethane.

In one example, wherein the one or more MHC class I epitopes and the one or more MHC class II epitopes are independently antigenic peptides derived from a nucleocapsid protein of an influenza virus.

In one example, wherein the one or more MHC class I epitopes are nucleocapsid protein₃₆₆₋₃₇₄ consisting of the amino acid sequence of SEQ ID NO: 1, and the one or more MHC class II epitopes are nucleocapsid protein₃₁₁₋₃₂₅ consisting of the amino acid sequence of SEQ ID NO: 2.

In one example, wherein the adjuvant comprises MPLA, CpG-ODN, poly(I:C), or variants of cyclic-dinucleotides.

In one example, wherein a lactide/glycolide ratio of the PLGA is about 40-60:60-40.

Another aspect of this invention is a method of neutralizing virus infection, comprising:

priming a subject in need thereof with an vaccine, wherein said vaccine comprises a polymeric hollow nanoparticle encapsulating one or more MHC class I epitopes; one or more MHC class II epitopes and an adjuvant.

In one example, wherein the polymeric hollow nanoparticle is substantially composed of poly(D,L-lactide-co-glycolide) (PLGA).

In one example, wherein a lactide/glycolide ratio of the PLGA is about 40-60:60-40.

In one example, wherein an intrinsic viscosity of the PLGA is about 0.15-0.25 d L/g.

In one example, wherein the one or more MHC class I epitopes and the one or more MHC class II epitopes are independently antigenic peptides derived from a nucleocapsid protein of an influenza virus.

In one example, wherein the one or more MHC class I epitopes are nucleocapsid protein₃₆₆₋₃₇₄ consisting of the amino acid sequence of SEQ ID NO: 1, and the one or more MHC class II epitopes are nucleocapsid protein₃₁₁₋₃₂₅ consisting of the amino acid sequence of SEQ ID NO: 2.

In one example, wherein the adjuvant comprises MPLA, CpG-ODN, poly(I:C), or variants of cyclic-dinucleotides.

In one example, the method is further comprising:

boosting the subject with the vaccine.

In one example, wherein the priming step and the boosting step is by at least one mode selected from the group consisting of parenteral, subcutaneous, intramuscular, intravenous, intra-articular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

In one example, wherein the priming step and the boosting step are by subcutaneous or intranasal.

### BRIEF DESCRIPTION OF THE DRAWINGS

FIG. 1 is CryoEM visualization of peptide-based influenza nanoparticle vaccine.

FIG. 2 is peripheral subcutaneous priming with PLGA nanoparticles encapsulating peptides and CpG induces robust T cell immunity. (A) Schematic representation of the experimental protocol for PLGA (OVA_{I/II}+CpG) titration. WT (Thy1.2) mice were co-transferred with CFSE-stained naïve Thy1.1⁺CD8⁺OT-I and Thy1.1⁺CD4⁺OT-II cells one day before immunization. At day 0, mice were immunized with PBS control, empty PLGA control, indicated doses of PLGA (OVA_{I/II}+CpG), or crude mixture of OVA_{I/II}+CpG. At day 7 post immunization, mice were sacrificed for analysis of the proliferation and INF-γ production of Thy1.1⁺OT-II and Thy1.1⁺OT-I cells in the spleen and lymph node. Representative flow cytometric plots displaying the proliferation (B) and INF-γ production (D) of Thy1.1⁺OT-II and Thy1.1⁺OT-I cells in spleens and inguinal LNs were shown. (C and E) Summary bar graphs for the mean percentages and cell numbers with SE of Thy1.1⁺OT-II and Thy1.1⁺OT-I T cells in spleens and dLNs (n≥6 mice per group complied from 3 independent experiments).

FIG. 3. Peripheral or local priming by PLGA nanoparticles encapsulating peptides and CpG causes minimal systemic adverse effects and pulmonary immunopathology. (A) The proportional body weight change of recipient WT mice from FIG. 1A were monitored at the indicated days post immunization. (B and C) A day 7 post-immunization inguinal LNs (B) and spleens (C) were measured for organ weight (C). (D) Harvested spleens were also photographed. (E) Pulmonary histological changes in WT mice receiving peripheral priming (s.c.) and local-boosting (i.n.) by the indicated vaccines at a one-month interval. At day 3 post secondary immunization, mice were sacrificed for analysis by H&E stain. Stained lung sections were examined by the light microscope. Scale bar, 200 μm. Data were pooled from 2^{˜}3 independent experiments. Individual organ weight of immunized mice for spleen and dLN with means plus SE (n≥4 mice per group). **, p≤0.01; ***, p≤0.001. (one-way ANOVA)

FIG. 4. The immunogenicity of nanoparticles in lungs.

FIG. 5. The immunogenicity and protectivity of nanoparticles by different vaccination strategies. (A) Schematic representation of the experimental protocol. C57BL/6 mice received primary s.c. (OVA_{I/II} with CpG, and 500 μg of PLGA) or i.n. (300 μg of PLGA) immunization. At day 28 post primary immunization, mice received secondary immunization with indicated vaccine formulas through i.n. or s.c. Mice were infected with 5×10⁵ PFU of HKx31-HA-OVA_{I/II} at day 56 post primary immunization (28 days after secondary immunization), and monitored the survival rate (B) and body weight change (C). (D) Lung viral loads were analyzed at day 5 post-HKx31-HA-OVA_{I/II} infection. Data are individual viral loads with means plus SE (n≥4 mice per group compiled from 3 independent experiments). (E) Individual percentages of virus-specific IFN-γ-producing T cells for spleens and dLNs with means plus SE (n≥4 mice per group). *, p≤0.05; **, p≤0.01; ***, p≤0.001. (Log-rank test for the survival rate and Student T test for percentages of IFN-γ production).

FIG. 6. The immunogenicity and protectivity of nanoparticles with NP₃₆₆₋₃₇₄/NP₃₁₁₋₃₂₅ by peripheral priming and local boosting. The experimental protocol was similar to FIG. 3A, except that OVA_{I/II} peptides and HKx31-HA-OVA_{I/II} were replaced by NP_{I/II} and PR8 (110 PFU), respectively. (A) The body weight and (B) survival rates of PR8-infected mice immunized by empty (circle), NP_{I}/NP_{II} peptides alone (black triangle), NP_{I}/NP_{II} peptides with CpG adjuvant (white triangle), PLGA (NP_{I/II}) (black square), or PLGA (OVA_{I/II}+CpG) (white square). (C and D) Lung viral load was analyzed at day 3^{˜}7 post PR8 infection. Data are individual viral loads with means plus SE (n≥5 mice per group compiled from 2 independent experiments). (E) NP_{I}-specific CD8 and NP_{II}-specific CD4 T cell immunity of spleens, dLNs, and lungs were analyzed at day 7 post-PR8 infection. Individual percentages of NP_{I}-specific IFN-γ-producing CD8 T cells and NP_{II}-specific IFN-γ-producing CD4 T cells for spleens (n≥5 mice per group complied from 2 independent experiments), dLNs (n≥5 mice per group complied from 2 independent experiments) and lungs with means plus SE (n≥5 mice per group complied from 2 independent experiments). (F) NP_{I}-specific IFN-γ-producing CD8 T cells and NP_{II}-specific IFN-γ-producing CD4 T cells in lungs. Data are individual cell numbers with means plus SE (n≥5 mice per group complied from 2 independent experiments). *, p≤0.05; **, p≤0.01; ***, p≤0.001. (Fisher's exact test for survival rate and Student T test for percentages of IFN-γ production).

FIG. 7. The cross-protectivity of nanoparticles with NP₃₆₆₋₃₇₄/NP₃₁₁₋₃₂₅ by peripheral priming and local boosting. The experimental protocol was similar to FIG. 4A, except that PR8 was replaced by HKx31 or WSN. (A and B) The body weight of HKx31-(A) or WSN-(B) infected mice immunized by empty (black circle), and NP_{I}/NP_{II} peptides with CpG adjuvant (white square). (C and D) Lung viral load was analyzed at day 7 post infection of HKx31 (C) or WSN (D). Data are individual viral loads with means plus SE (n≥5 mice per group compiled from 2 independent experiments). ***, p≤0.001. (Student T test).

FIG. 8. The comparison of memory T cell populations induced by nanoparticles with the peripheral priming/local boosting and the local priming/local boosting strategy. (A) WT (Thy1.2) mice were transferred with naïve Thy1.1⁺CD8⁺OT-I cells one day before immunization, and immunized by the indicated individual protocols. Memory T cells were analyzed at 28 days post secondary immunization. (B and C) Spleen samples were gated on Thy1.1⁺CD8⁺CD44⁺ cells, and determined for the frequency and total number of Tcm (CD62L⁺KLRG-1⁻) and Tem (CD62L⁻KLRG-1⁺) cells (n≥7 mice per group complied from 3 independent experiments). (D and E) In vivo CD3 antibody staining and ex vivo CD8 antibody staining were performed to measure Trm cells. Lung samples were gated on Thy1.1⁺CD3e⁻CD8⁺CD44⁺CD62L-KLRG-1⁻ cells, and analyzed for the percentage and total number of CD69⁺, CD103⁺, and CD69⁺CD103⁺ cells. (n≥7 mice per group complied from 3 independent experiments). *, p≤0.05; **, p≤0.01; ***, p≤0.001. (Student T test)

FIG. 9. The durability of lung-resident memory T cells elicited by nanoshell vaccines with different vaccination strategies. (A) WT (Thy1.2) mice were transferred with naïve Thy1.1⁺CD8⁺OT-I cells one day before immunization, and immunized by the indicated individual protocols. Memory T cells were analyzed at 56 days (2 months) or 84 days (3 months) post secondary immunization. (B) At 56 days after secondary immunization, spleen samples were analyzed for the frequency and total number of Tcm (CD62L⁺KLRG-1⁻) and Tem (CD62L⁻KLRG-1⁺) cells (n=3 mice per group). (C) At 56 days after secondary immunization, lung samples were gated on Thy1.1⁺CD3e⁻CD8⁺CD44⁺CD62L⁻KLRG-1⁻ cells, and analyzed for the percentage and total number of Trm cells, defined as CD69⁺CD103⁺ cells. (n=3 mice per group). (D) At 84 days after secondary immunization, spleen samples were analyzed for the frequency and total number of Tcm and Tem cells (n=3 mice per group except 2 mice for the PBS group and the s.c./i.n. NS(OVA+CpG) group). (E) At 84 days after secondary immunization, lung samples were analyzed for Trm cells. (n=3 mice per group except 2 mice for the PBS group). *, p≤0.05; **, p≤0.01. (Student's t-test)

FIG. 10. Uptake and tracking of nanoparticles in lungs and dLNs at 12 hours post immunization

FIG. 11. Uptake and tracking of nanoparticles in lungs and dLNs. (A) Lung samples were gated on macrophage (SSC^{High}CD11c⁺MHC-II^{Low}F4/80⁺) and dendritic cells (SSC^{Low}CD11c⁺MHC-II^{High} CD103⁺ and SSC^{Low}CD11c⁺MHC-II^{High} CD11b⁺), and determined for (B) the representative flow cytometric plot of PLGA (AF555) uptake at 24 hr. (C) The percentages of macrophage (SSC^{High}CD11c⁺MHC-II^{Low}F4/80⁺) and dendritic cells (SSC^{Low}CD11c⁺MHC-II^{High} CD103⁺ and SSC^{Low}CD11c⁺MHC-II^{High} CD11b⁺) in lungs at 24 hr (n=4 mice per group complied from 2 independent experiments). (D) t-SNE map of different subset of dendritic cells (AF555⁺) colored by FlowSOM metaclusters in lungs at 24 hr. Date are downsampled to 1×10⁶ cells/mice (form 3 mice/group), and representative heatmap statistic is 1 mouse per group. The lower panel t-SNE maps were gated by AF555⁺ cell. The color bar represents the expression levels of indicated proteins in PLGA-taking (AF555⁺) cells. (E) The percentages of CD86+ or IFN-γ-producing cells in PLGA-taking (AF555⁺) CD11c⁺CD103⁺ and CD11c⁺CD11b⁺ dendritic cells of lungs at 24 hr (n≥3 mice per group complied from 2 independent experiments). (F) t-SNE map of different subset of dendritic cells (AF555⁺) colored by FlowSOM metaclusters for LN at 24 hr. Data are downsampled to 1×10⁶ cells/mice (form 3 mice/group, contour), and representative heatmap statistic is 1 mouse per group. Color bar means the proportion of PLGA uptake cells. (G) The mean fluorescent (AF555) intensity (MFI) of PLGA-taking (AF555⁺) in CD11c⁻ and CD11c⁺ cells of LNs at 24 hr (n≥3 mice per group complied from 2 independent experiments). (H) The individual cell numbers of AF555⁺CD11c⁺MHC-II⁺CD103⁺ and AF555⁺CD11c⁺MHC-II⁺CD11b⁺ in LNs at indicated time points (n≥3 mice per group complied from 2 independent experiments). *, p≤0.05; **, p≤0.01; ***, p≤0.001, ****, p≤0.0001. (Student's t-test)

FIG. 12. Uptake and tracking of nanoparticles in lungs and dLNs at 48 hours post immunization.

FIG. 13. CD11c-positive APCs are required for stimulation of T cells by nanoparticle peptide vaccines. (A) Schematic representation of the experimental protocol. Mice received PBS or DT depletion two days before immunization, and then were immunized with either PLGA (OVA_{I/II}) or PLGA (OVA_{I/II}+CpG) through i.n. One day after immunization, mice were co-transferred with CFSE-stained Thy1.1⁺CD8⁺OT-I and Thy1.1⁺CD4⁺OT-II cells, sacrificed for analysis at day 3 post immunization. (B) Representative flow cytometric plots displaying the efficacy of CD11c⁺ cells depletion at day 5 post DT treatment. (C) Representative flow cytometric plots displaying the proliferation of OT-I and OT-II. (D and E) Individual percentages (D) and cell numbers (E) of proliferating CD4⁺OT-II and CD8⁺OT-I cells in dLNs (n≥6 mice per group for 3 experiment). *, p≤0.05; **, p≤0.01; ***, p≤0.001. (Student's t-test).

### DETAILED DESCRIPTION

Pre-existing cross-reactive T cell immunity against newly emerging influenza viruses has been a strong support for the development of T cell-based universal influenza vaccines (5-7). A very recent study discovered highly conserved CD8+ T-cell epitopes across influenza A, B and C viruses presented by dominant class I HLAs further suggested the utility of peptide-based T cell vaccines against diverse influenza virus strains and subtypes(8). Although viral vectors have been shown to elicit protective T cell immunity against IAVs, most nonviral peptide vaccine carriers have unsatisfactory T cell-stimulating ability, and fail to achieve full antiviral protection. In this study, we demonstrated our novel biocompatible hollow PLGA nanoparticles with co-encapsulation of only two epitope peptides and CpG elicited robust antigen-specific CD4 and CD8 T cell immunity, and protected against lethal IAVs of different strains (PR8, WSN and HKx31) and subtypes (H1N1, and H3N2). This is a proof of concept that with appropriate choice of T-cell epitope peptides and an adjuvant, this novel non-replicating nanoparticle peptide vaccine, when utilized in the peripheral priming and local boosting vaccination strategy, can induce robust and highly protective T cell immunity against IAV infections.

The vaccine in this application comprises: a polymeric hollow nanoparticle encapsulating one or more MHC class I epitopes; one or more MHC class II epitopes; and an adjuvant. The polymeric hollow nanoparticle is composed of poly(D,L-lactide-co-glycolide) (PLGA). Preferably, PLGA is carboxy terminated. Preferably, the ratio of lactic:glycolide of PLGA is about 40-60:60-40, and more preferably is 50:50. Preferably, viscosity of PLGA is 0.05-0.35 dL/g, and more preferably is 0.15-0.25 dL/g. Biodegradable PLGA nanoparticles are a suitable vaccine carrier for the potent immunogenicity and excellent safety profile. Our novel PLGA nanoparticle is advantageous for its small size. Preferably, the size of nanoparticle is around 50-200 nm, and more preferably is 100-180 nm, and much preferably is 150-160 m). Previous studies have shown that the size of nanoparticles affects their uptake efficiency by APCs (20). The small size of our nanoparticle renders it the superior uptake by DCs and the consequent T cell priming activity. The one or more MHC class I epitopes and the one or more MHC class II epitopes are independently antigenic peptides derived from a protein of a virus. The virus is preferably selected from influenza A virus, influenza B virus and influenza C virus. Preferably, the virus is influenza A virus. There are two groups for protein of the virus: structural proteins and non-structural proteins. Preferably, the peptides of the application is derived from structural proteins, comprising haemagluttinin (HA), neuraminidase (NA), membrane protein (M) and nucleocapsid protein (NP). More preferably, the peptides is derived from nucleocapsid protein. In one example, the one or more MHC class I epitopes are nucleocapsid protein₃₆₆₋₃₇₄ consisting of the amino acid sequence of SEQ ID NO: 1. In one example, the one or more MHC class II epitopes are nucleocapsid protein₃₁₁₋₃₂₅ consisting of the amino acid sequence of SEQ ID NO: 2. The adjuvant in this application is selected from the group consisting of Alum, MF59, AS01, AS03, AS04, Flagellin, CAF01, IC31, ISCOMATRIX, MPLA, CpG-ODN, poly(I:C), and variants of cyclic-dinucleotides. Preferably, the adjuvant comprise MPLA, CpG-ODN, poly(I:C), or variants of cyclic-dinucleotides. More preferably, the adjuvant is CpG-ODN.

This application further provides a method of manufacturing a vaccine, said vaccine comprising a polymeric hollow nanoparticle encapsulating one or more MHC class I epitopes, one or more MHC class II epitopes, and an adjuvant, comprising:

emulsifying a first solution comprising one or more MHC class I epitopes, one or more MHC class II epitopes and an adjuvant in a solvent comprising poly(D,L-lactide-co-glycolide) (PLGA);

sonicating the emulsion; and

purifying the polymeric hollow nanoparticle in the emulsion.

The first solution is alkaline buffer. The alkaline buffer comprises sodium bicarbonate, potassium persulphate or the combination thereof. Preferably, the alkaline buffer only comprises sodium bicarbonate. The concentration of sodium bicarbonate is 100-300 mM, and preferably is 150-250 mM, and more preferably is 200 mM. The volume of sodium bicarbonate is 20-80 uL, and preferably is 50 uL. The polymeric hollow nanoparticle, the one or more MHC class I epitopes, the one or more MHC class II epitopes, and the adjuvant are set forth. The concentration of the one or more MHC class I epitopes and the one or more MHC class II epitopes is 1.0-5.0 mg/mL, preferably is 2.0-4.0 mg/mL and more preferably is 3.3 mg/mL. The concentration of the adjuvant is 1.0-4.0 mg/mL, preferably is 2.0-3.0 mg/mL and more preferably is 2.5 mg/mL.

The solvent comprises dichloromethane. Preferably, the solvent only comprises dichloromethane. The volume of dichloromethane is 200-800 uL, and preferably is 500 uL. The concentration of the PLGA is 20-80 mg/mL, preferably is 35-65 mg/mL and more preferably is 50 mg/mL.

The first emulsion for emulsifying the first solution in the solvent use an Ultrasonic Probe Sonicator under the pulse mode with 35-65% amplitude and on-off durations of 0.5 and 2.5 s for 0.5-2.5 min, and preferably the pulse mode with 40% amplitude and on-off durations of 1 and 2 s for 1 min.

For purification of the polymeric hollow nanoparticle in the first emulsion, the nanoparticles were collected and purified from unencapsulated adjuvant and peptides through centrifugal wash using an Amicon Filter (MWCO 100,000 Da).

In one embodiment, the method of manufacturing a vaccine further comprising:

adding a second solution to the emulsion after the sonicating step.

The second solution is phosphate buffer. The concentration of phosphate buffer is 0.1-10 mM, and preferably is 0.5-3.0 mM, and more preferably is 1 mM. The volume of phosphate buffer is 1 mL, and preferably is 5 mL. The pH value of phosphate buffer is pH 6.-7.5, and preferably is pH 7. The second emulsion for emulsifying the second solution in the product of the first emulsion use an Ultrasonic Probe Sonicator under the pulse mode with 15-45% amplitude and on-off durations of 0.5 and 2.5 s for 1-3 min, and preferably the pulse mode with 30% amplitude and on-off durations of 1 and 2 s for 2 min. at 30% amplitude with on-off durations of 1 and 2 s for 2 min.

In one embodiment, the method of manufacturing a vaccine further comprising:

pouring the emulsion to water after the adding step; and

evaporating the solvent from the emulsion.

For solvent evaporation, the second emulsion was subsequently poured to 2-16 mL of water and heated at 50-60° C. under gentle stirring in a fume hood for 15-45 min. Preferably, solvent evaporation is proceed by 8 mL of water and heated at 40° C. under gentle stirring in a fume hood for 30 min.

After the purification, the resulting nanoparticles were characterized and frozen in 10% sucrose at −20° C.

The results showed that compared to the crude mixture of peptides and CpG, this novel PLGA nanoparticle vaccine with peptides and CpG elicited robust antigen-specific CD4 and CD8 T cell responses, but caused negligible systemic adverse inflammatory effect, which was evident by the nearly normal-sized spleens of immunized mice. We calculated the doses of encapsulated peptides and CpG (500 μg nanoparticle), which were only about one-fifth peptides and one-fortieth CpG of the crude mixture. The effective uptake by APCs may also facilitate trapping of nanoparticles at local immunization sites to minimize systemic spread and adverse inflammatory responses.

T cell vaccine usually does not provide sterilizing immunity, but is considered to only reduce the severity of disease. Recently, Trm cells have been recognized as the first-line defense against invading pathogens and exhibit innate-like and near-sterilizing immunity (19). Trm cells in lungs are shown to be critical for protection against IAV infection (17, 18). In addition, vaccination routes influence the generation of protective T cell immunity (21). We adopted the peripheral subcutaneous priming and local intranasal boosting immunization strategy, and demonstrated that local boosting was required for the protectivity against IAV, which was associated with establishment of robust Trm cells in lungs. This application also provides a method of neutralizing virus infection, comprising:

priming a subject in need thereof with an vaccine, wherein said vaccine comprises a polymeric hollow nanoparticle encapsulating one or more MHC class I epitopes; one or more MHC class II epitopes and an adjuvant.

The polymeric hollow nanoparticle, the one or more MHC class I epitopes, the one or more MHC class II epitopes, and the adjuvant are set forth.

The method of neutralizing virus infection, further comprising:

boosting the subject with the vaccine.

The priming step and the boosting step is by at least one mode selected from the group consisting of parenteral, subcutaneous, intramuscular, intravenous, intra-articular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

Preferably, priming step is by subcutaneous or intranasal. Preferably, the boosting step is by subcutaneous or intranasal. Preferably, the boosting step is by intranasal.

Nevertheless, Trm cells in lung are not always stable, but gradually decline along with time (22). Recently, Slutter et al. reported that circulatory Tem cells served as a memory T cell pool for replenishment of Trm cells in lungs (23). We showed, compared to the local priming and local boosting immunization strategy, peripherally priming and local boosting elicited significantly more circulatory Tem cells, but they both induced similar levels of robust Trm cells.

Nonviral vector peptide vaccines that are intended to elicit T cell immunity against viral infections have generated disappointing levels of protection because of their poor immunogenicity(3). Surprisingly, our CpG adjuvanted nanoparticle peptide vaccines, with only two class I and class II MHC-restricted peptides (NP366-374/NP311-325) derived from authentic influenza nucleoprotein were able to confer full protection against different influenza virus strains and subtypes. This result strongly argues that non-replicating nanoparticle peptide vaccines, when given in an optimal vaccine formula and an immunization strategy, can induce nearly sterilizing T cell immunity against IAV infection. Of note, choice of appropriate peptides as immunogens is important for successful protection. We found that mice immunized by nanoparticles with NP366-374/NP311-325 cleared influenza virus much faster than mice immunized by nanoparticles with OVAI/OVAII, although both groups of mice achieved 100% survival after lethal IAV challenge. The former group suppressed lung viral loads to undetectable levels by day 7 post infection, whereas the latter group only achieved around 10-fold reduction of replicating viruses. Previous studies have pointed out the expression abundance and timing of viral antigens in regards with the viral replication cycle determine the hierarchy of T cell responses and the resultant viral control(24, 25). For the recombinant influenza virus PR8-OVAI/OVAII, OVAI/OVAII peptides are co-expressed with NA protein following influenza virus infection. Therefore, the differential protectivity of these two nanoparticle peptide vaccines may be partially explained by the distinct expression patterns of NP and NA, leading to differential protectivity.

DCs in lungs play an important role in priming and activating T cells(26). Because of the essential role of local boosting with nanoparticles in inducing protective T cell immunity in lungs, it is reasonably assumed that pulmonary DCs are the main cell population targeted by our nanoparticle vaccines via intranasal administration(27, 28). By the tracking experiments with the nanoparticle-packaged small fluorescent molecule, we showed nanoparticles were efficiently taken by CD11c+ macrophages and DCs, and CD11c+CD103+ DCs were the main population for migration to dLNs. Consistently, the prior study showed CD11c+CD103+ migratory DCs are the main cell population that carries influenza viral antigens to dLNs, where they prime antigen-specific CD4 and CD8 T cells(29). Interestingly, specific depletion of CD11c+ cells by DT dramatically reduced the proliferation of T cells stimulated by nanoparticle vaccines, further supporting that CD11c+ APCs were responsible for the priming activity of the nanoparticle vaccines. In addition, we also demonstrated that the CpG adjuvant promoted the maturation of DCs, which was correlated with the better protectivity of nanoparticle-induced T cell immunity.

In summary, the findings in this study prove that, with appropriate nanoparticle design, antigenic peptides, adjuvants and immunization strategy, non-proliferating nanoparticle-packaged peptide-based T cell vaccines, like ours, are able to confer robust cross-protective T cell immunity against heterosubtypic and distantly related IAVs, a critical step toward the development of universal T cell-based vaccine.

### Mice

All mouse experiment protocols were approved by the Laboratory Animal Committee of National Taiwan University College of Medicine (NTUCOM). C57BL/6 wild-type mice (Thy1.2) were purchased from the National Laboratory Animal Center in Taiwan. Thy1.1/1.1×OT-I, Thy1.1/1.2×OT-I, and Thy1.1/Thy1.2×Foxp3^{gfp}×OT-II mice were generated by cross-breeding the indicated mouse lines in a C57BL/6 background by ourselves, and were maintained in the Laboratory Animal Center of NTUCOM. All mice used in this application were 6^{˜}8 week-old female mice. (Note: transgenic OT-I cells can specifically recognize MHC class I-restricted OVA₂₅₇₋₂₆₄, and OT-II cells can specifically recognize MHC class II-restricted OVA₃₂₃₋₃₃₉.)

### Viruses and Quantification of Viral Titers

HKx31-OVA_{I/II} (H3N2) was generated as previously describe (33), and stored at −80° C. Virus was diluted with PBS to the indicated doses for infection. Mice were anesthetized by intraperitoneal injection of a mixture of xylazine and tiletamine hypochloride and zolazepam hypochloride, and then infected with 20 μl of viral suspension via the intranasal route. IAV-infected mice were sacrificed on day 5 post-infection. Lungs were isolated and homogenized in 1 ml infection medium consisting of DMEM with NEAA, sodium pyruvate, and bovine serum albumin. Replicative virus titers were determined by the plaque assay. Briefly, 8.5×10⁵ MDCK cells/well were seeded in six-well plates. On the next day, serial tenfold dilutions of virus suspensions (100 μl) were inoculated and cultured at 37° C. for 1 hour. Agar medium (infection medium with 0.3% agarose) was then added to each well, and incubated at 37° C. for 2^{˜}4 days according to the virus strain. Cells were then fixed with 2% paraformaldehyde for at least 2 hours, and stained with 0.1% crystal violet in 75% ethanol.

### PLGA Nanoparticles

All PLGA nanoparticles in this application including empty PLGA, P(O), and P(O+C) were synthesized by the double emulsion method.

To prepare the peptide-based influenza vaccine, peptide antigens derived from influenza virus nucleoprotein, including NP₃₆₆₋₃₇₄ MHC I epitope and NP₃₁₁₋₃₂₅ MHC II epitope, were combined with a TLR9 agonist CpG-ODN 1826. To maximize the solubility of the peptide antigens and the immunologic adjuvant, 200 mM of sodium bicarbonate was adopted for the solubilization. To prepare the nanoparticle vaccine, 50 uL of 200 mM sodium bicarbonate solution containing 2.5 mg/mL of CpG, 3.3 mg/mL of NP₃₆₆₋₃₇₄, and 3.3 mg/mL of NP₃₁₁₋₃₂₅ was first emulsified in 500 uL of dichloromethane containing 50 mg/mL poly(lactic-co-glycolide acid) using an Ultrasonic Probe Sonicator under the pulse mode with 40% amplitude and on-off durations of 1 and 2 s for 1 min. The poly(lactic-co-glycolide acid), PLGA is carboxy terminated with the ratio of lactic:glycolide being 50:50, and viscosity thereof is 0.15-0.25 dL/g. The first emulsion was subsequently added to 5 mL of 1 mM phosphate buffer (pH 7), which was then probe sonicated at 30% amplitude with on-off durations of 1 and 2 s for 2 min. The emulsion was subsequently poured to 8 mL of water and heated at 40 C under gentle stirring in a fume hood for solvent evaporation. Following 30 min of solvent evaporation, the nanoparticles were collected and purified from unencapsulated adjuvant and peptides through centrifugal wash using an Amicon Filter (MWCO 100,000 Da). The resulting nanoparticles were characterized and frozen in 10% sucrose at −20° C.

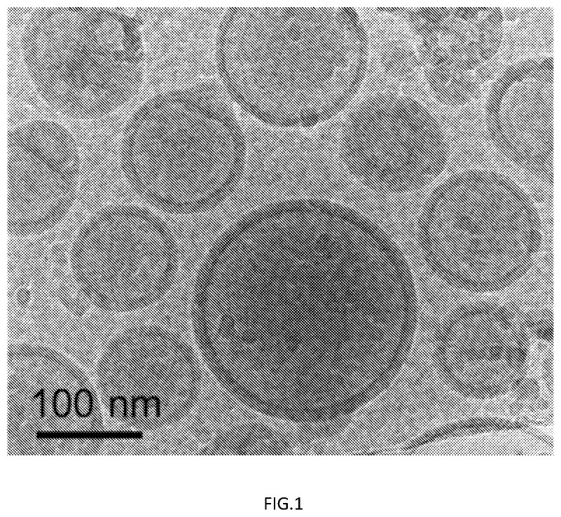

The nanoparticle vaccines have an average size of 152±5 nm and distinctive hollow structure upon examination under cryoEM (FIG. 1). A batch of 100 mg PLGA particles were prepared each time. For P(O), 100 mg PLGA contained 33 μg OVA_{I} and 37 μg OVA_{II}. For P(O+C), the same amounts of OVA peptides were encapsulated, with an addition of 25 μg CpG-ODN (Invivogen). The encapsulation efficiency is 50% for the CpG-ODN and the peptides, corresponding to 2.5 ug of CpG, 3.3 ug of NP₃₆₆₋₃₇₄, and 3.3 ug of NP₃₁₁₋₃₂₅ encapsulated in 1 mg of PLGA nanoparticle. Given that 1 mg of PLGA yields approximately 1×10¹² nanoparticles upon measurement by nanoparticle tracking analysis, each nanoparticle contains approximately 236 CpG, 1936 NP366-374 peptides, and 1125 NP311-325 peptides. Particles were diluted by 1×PBS, or ddH₂O supplemented with 10 mM disodium phosphate and 10% sucrose. All particles were shipped under 4° C., and stored at −80° C. for use within one week.

### Intravascular Staining

Mice were intravenously injected at the tail vein with 3 μg of anti-CD3e APC clone 145-2C11 (eBioscience) in 300 μl PBS, and sacrificed 11 minutes later. Cardiac puncture was performed, then mice were perfused with 20^{˜}25 ml of PBS. Indicated tissues were then harvested, isolated for single cells, and stained for surface markers for further analyzes by flow cytometers.

Dendritic Cell Isolation from the Lung and Lymph Node

Harvested mice lung and mediastinal LN were minced by scissors into 1 mm³ sections and digested with 0.5 mg/mL collagenase type IV in RPMI 1640 supplemented with 1% Glutamine-Penicillin-Streptomycin and 25 U/ml type IV DNase I under agitation at 37° C. for 30 minutes (LN) or 60 minutes (lung). Reaction was stopped by the addition of PBS supplemented with 2% FBS. Lung samples were dispersed by syringe fitted with a 18 G needle; LN samples were dispersed by 100 μl pipette tips. Cells were then passed through cell strainers, treated by RBS lysis buffer (eBioscience) if needed, and washed by PBS supplemented with 2% FBS for further staining.

### Cell Staining, Antibodies, and Flow Cytometry

Cells were washed twice with staining buffer (PBS containing 2% FBS), and stained for 30 minutes at 4° C. with the following antibodies: anti-Thy1.1 APC clone HIS51 (eBioscience), anti-Thy1.1 BV510 clone OX-7 (BioLegend), anti-CD4 PerCP-Cy5.5 clone RM4-5 (eBioscience), anti-CD8a PE-Cy7 clone 53-6.7 (eBioscience), anti-CD44 BV650 clone IM7 (BioLegend), anti-CD69 PE clone H1.2F3 (eBioscience), anti-CD103 BV421 clone 2E7 (BioLegend), anti-CD62L BUV737 clone MEL-14 (BD Biosciences), anti-KLRG-1 BUV395 clone 2F1 (BD Biosciences), anti-CD11c BB515 clone N418 (BD Biosciences), anti-CD11b BV711 clone M1/70 (BD Biosciences). When two or more BD Horizon Brilliant dyes were used, cells were stained in Brilliant Stain Buffer (BD Biosciences) to optimize staining conditions. For intracellular staining, cells were fixed and permeabilized (Cytofix/Cytoperm, BD Biosciences) after surface staining, and stained with anti-IFN-γ APC clone XMG1.2 (BD Biosciences). Flow cytometry was performed and analyzed using FACS Verse or LSR Fortessa.

### Statistical Analyses

Data are expressed as mean±standard error of mean (SEM). Continuous variables, including the percentage of antigen-specific T cell responses and lung viral titers, were analyzed by one-way ANOVA. Survival rates were analyzed by Log-rank (Mantel-Cox) test. A p value of <0.05 was considered statistically significant.

### PLGA Nanoparticles Co-Encapsulating Peptides and CpG Induce Robust Antigen-Specific T Cell Responses but Minimal Systemic Adverse Effects

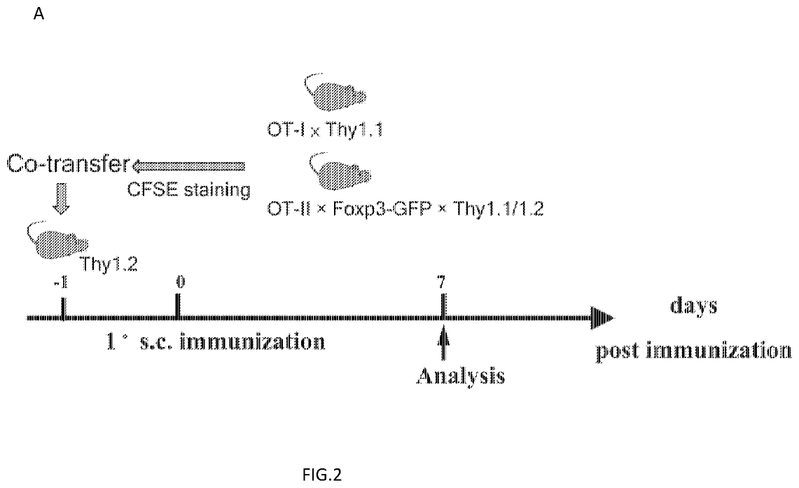

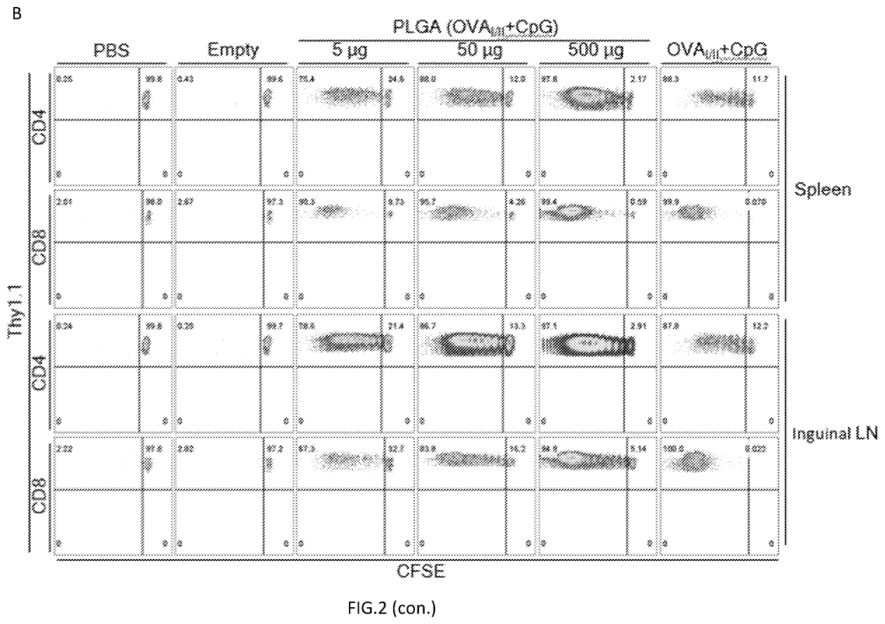

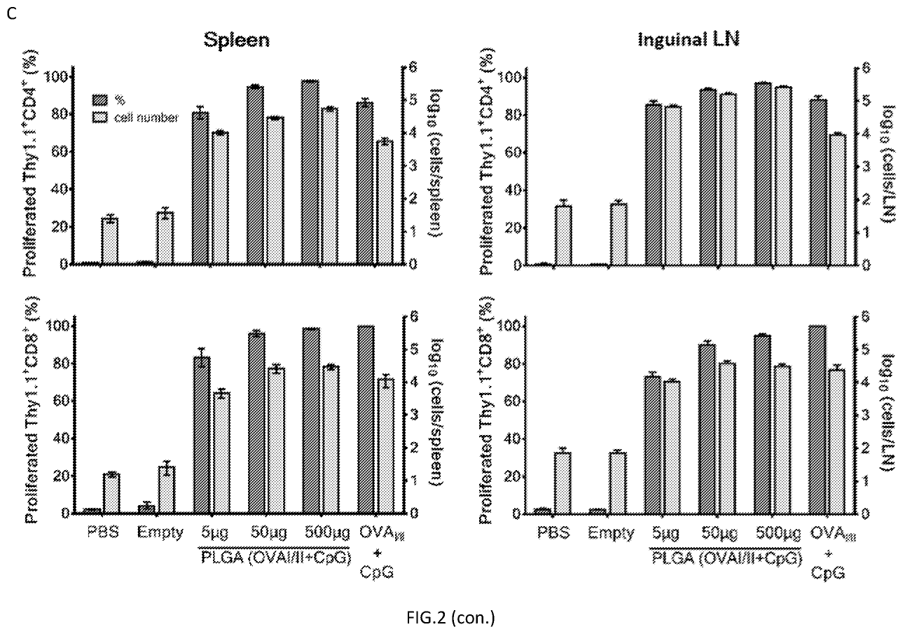

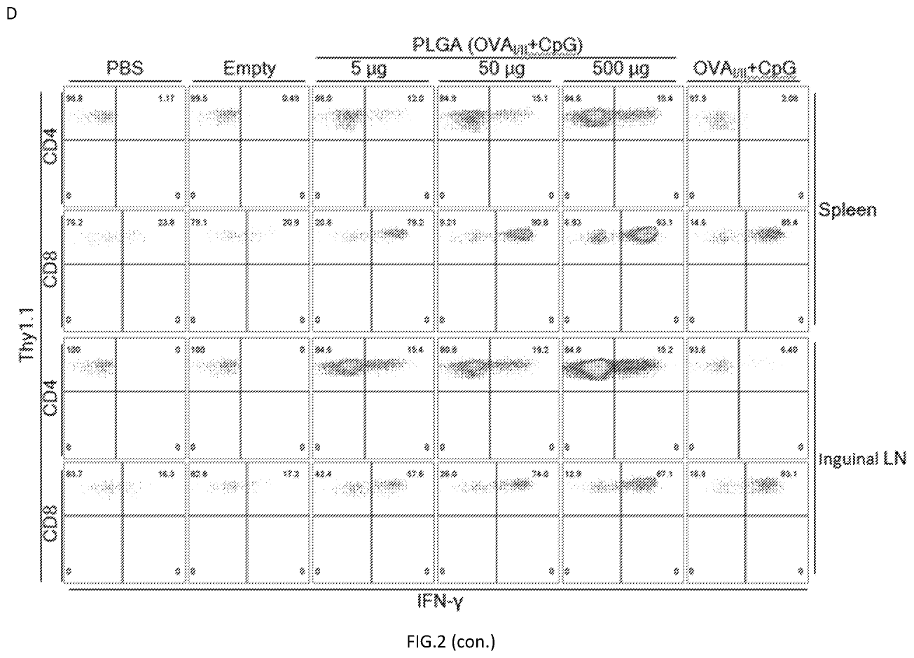

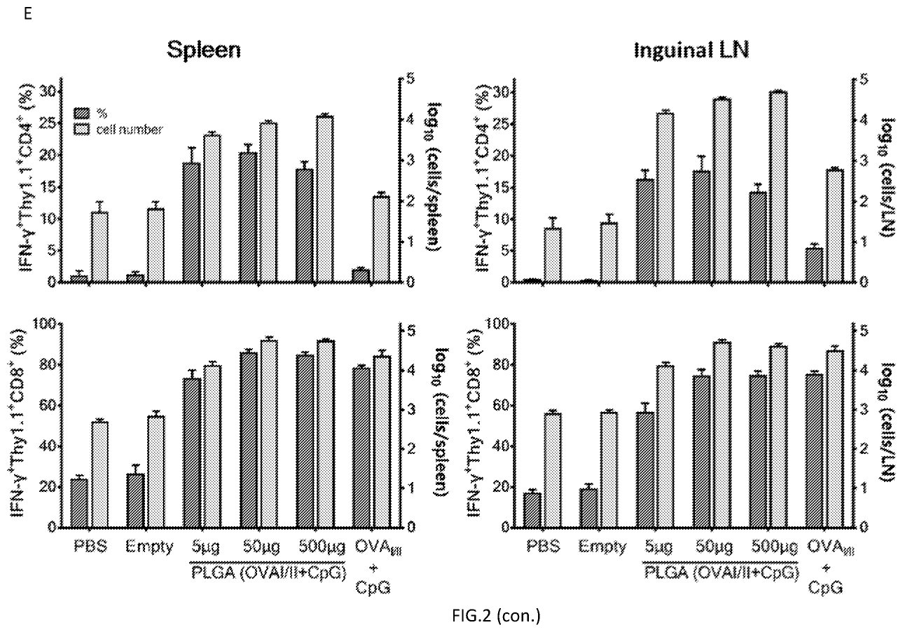

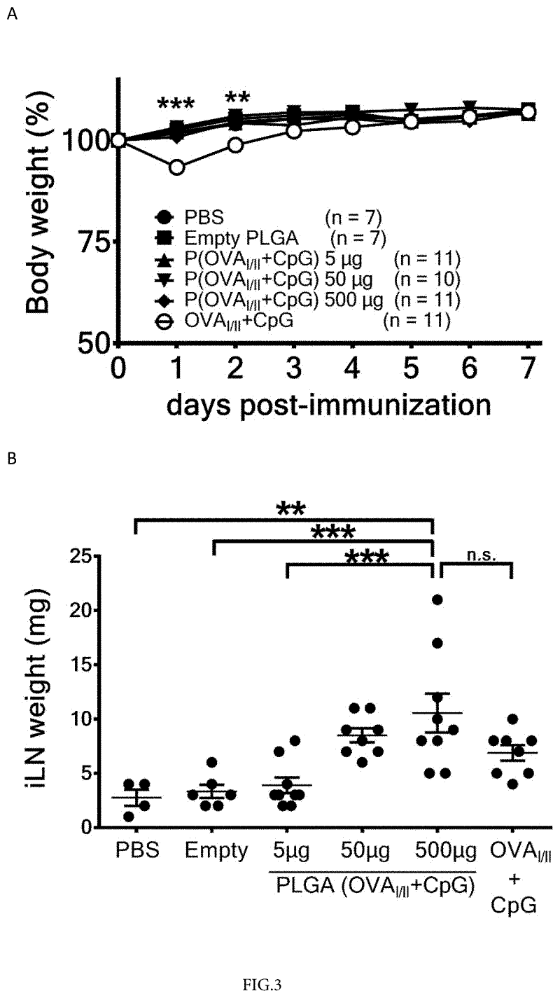

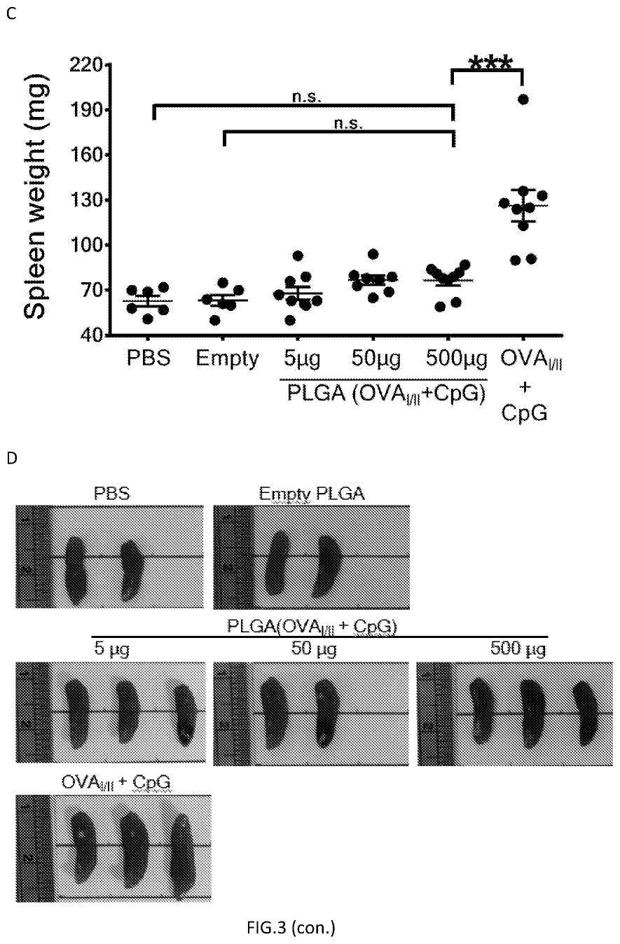

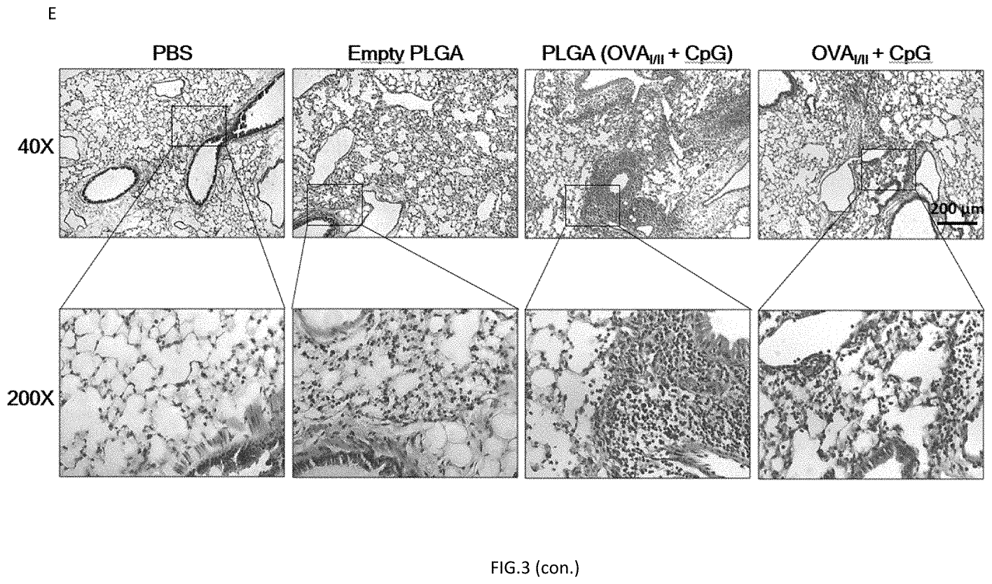

To investigate antigen-specific T cell responses, we used model antigenic ovalbumin peptides OVA₂₅₇₋₂₆₄ (OVA_{I})/OVA₃₂₃₋₃₃₉ (OVA_{II}) and their respective cognate OT-I/OT-II transgenic T cells. Our previous application has shown that CpG-adjuvanted peptide vaccines stimulate antigen-specific T cell immunity more effectively than unadjuvanted peptide vaccines(11). Recently, we have developed a novel PLGA nanoparticle vaccine carrier that is small (around 150-180 μM) and hollow and can efficiently co-encapsulate peptides and CpG. To determine whether the novel nanoparticle CpG-adjuvanted peptide vaccines induces stronger antigen-specific CD4 and CD8 T cell immunity than simple mixture of peptides and CpG, naïve wildtype (WT) Thy1.2^{+/+} mice were adoptively transferred with Thy1.1^{+/+} CFSE-stained OT-I and OT-II T cells, and were then subcutaneously (s.c.) immunized with titrated doses of PLGA nanoparticles that co-encapsulate OVA_{I}/OVA_{II} peptides and CpG (P(O+C)) or simple mixture of OVA_{I}/OVA_{II} peptides and CpG (O+C) (FIG. 2A). On day 7 after immunization, transferred OT-I and OT-II cells in the spleens and draining lymph nodes (dLNs) of vaccinated mice were analyzed by flow cytometry, which showed strong proliferation of OT-I and OT-II T cells, up to >90%, induced by P(O+C) in a dose-dependent manner (FIG. 2B, C). Compared with simple mixture O+C, 500 μg P(O+C), the maximal dose used, induced similar levels of CD8 T cell proliferation, but a significantly stronger CD4 T cell proliferation. In addition, mice that were s.c. immunized by 500 μg P(O+C) induced about 75% and 15% of the transferred CD8⁺ OT-I T cells and CD4⁺OT-II T cells for IFN-γ production, significantly higher than those of the PBS and empty PLGA control groups (FIG. 2D, E). Furthermore, compared to O+C, 500 μg P(O+C) caused a significantly higher proportion of IFN-γ-producing OT-II cells in both spleens and dLNs (17.7% versus 1.9%, and 14.2% versus 5.3% respectively). Of note, the amount of CpG-ODN, OVA_{I} and OVA_{II} encapsulated in PLGA nanoparticles were approximately 40-, 6-, and 5.4-fold less than those in the O+C group. Also, no obvious weight loss was noted in all PLGA nanoparticle peptide-vaccinated mice, but significant weight loss was measured on the very next day in mice administered with O+C (FIG. 3A). On day 7 post-immunization, while inguinal draining LNs in mice immunized by 500 μg P(O+C) were significantly heavier than the control groups, there was no difference in the size and weight of spleens between all PLGA-vaccinated mice and control groups (FIG. 3B, C). In contrast, the spleens of (O+C)-vaccinated mice were significantly heavier than those of the rest of groups (FIG. 3C, D).

Local Intranasal Priming with Nanoparticle Peptide Vaccines in Lung Induces Robust Antigen-Specific T Cell Responses and Tolerable Immunopathology

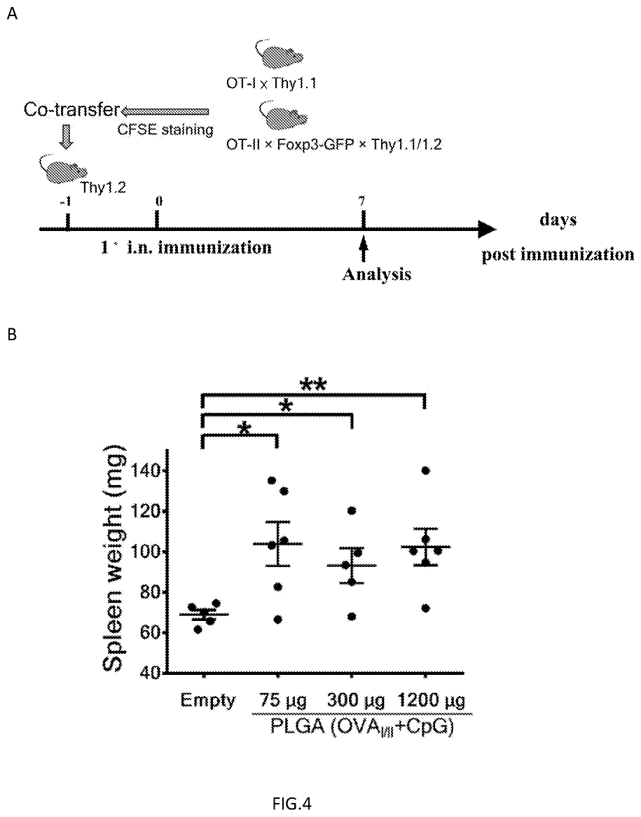

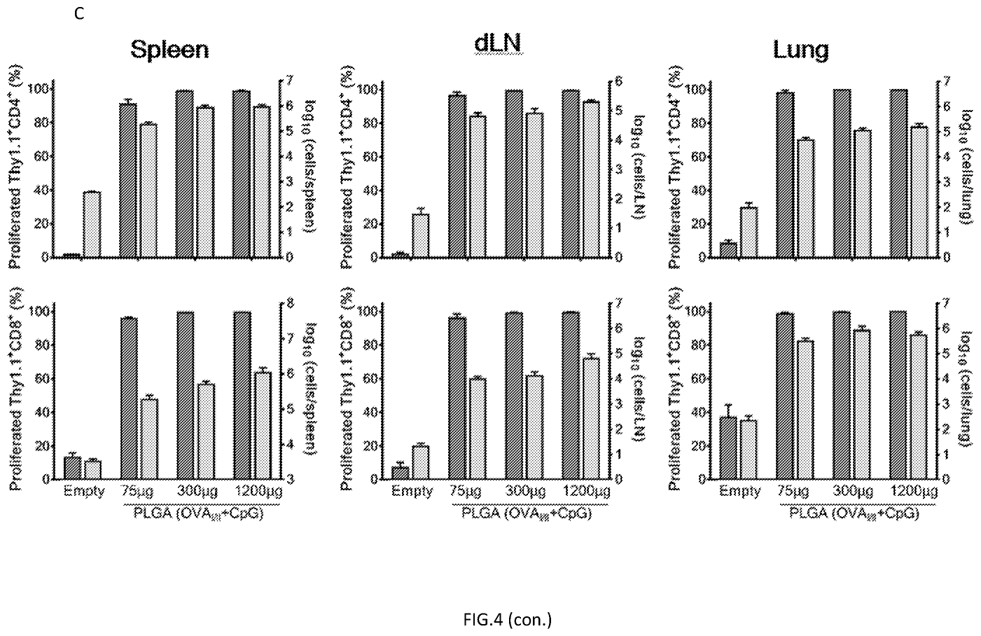

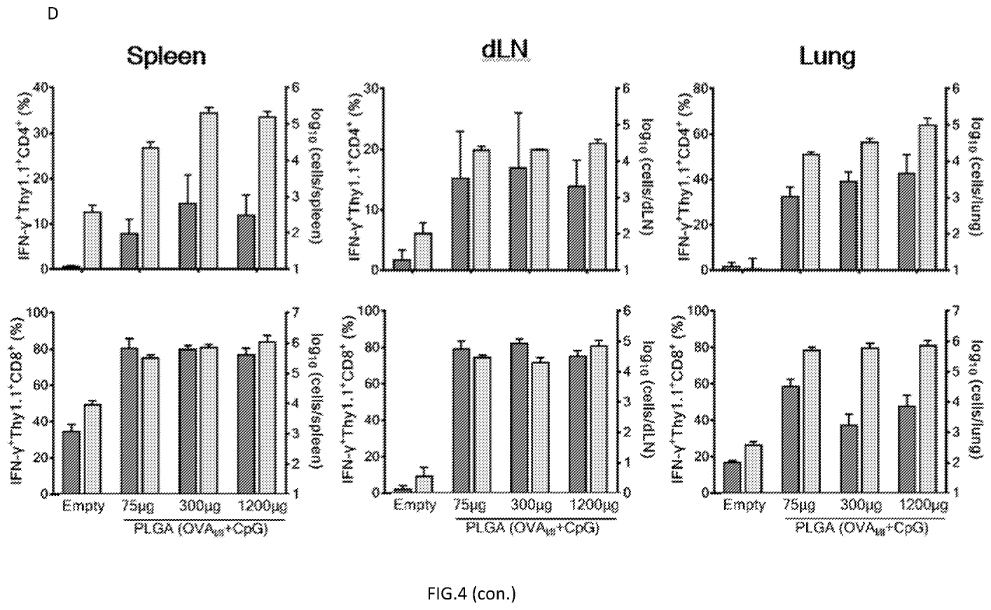

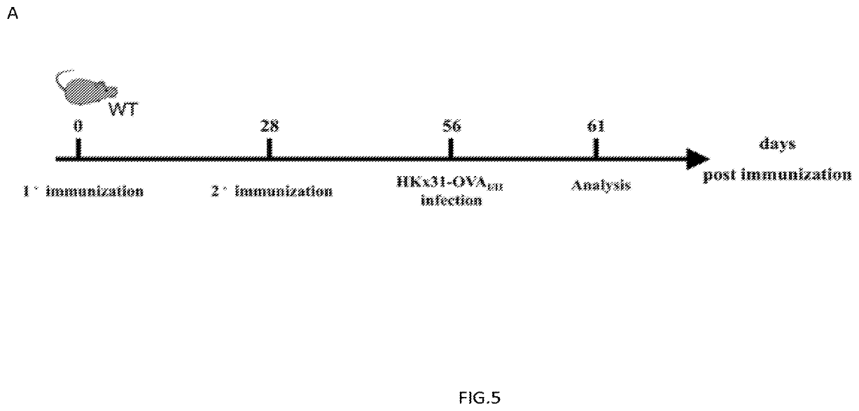

We next tested the doses of P(O+C) via intranasal administration. Naïve Thy1.2^{+/+} mice were adoptively transferred with Thy1.1^{+/+} CFSE-stained OT-I and OT-II cells and then intranasally (i.n.) instilled with titrated doses of P(O+C) (FIG. 4A). On day 7 post-immunization, mice were sacrificed and the lungs, mediastinal LNs (MedLN) and spleens were analyzed. P(O+C) stimulated significantly stronger T cell activation than the empty PLGA control in a dose-dependent manner (FIGS. 4B and C). While 75 μg P(O+C) did not cause weight loss throughout the seven-day-period monitored, 300 μg led to a mild drop of body weight on day 5 post-immunization, and 1200 μg i.n. P(O+C) resulted in the greatest extent of weight drop from day 4 after the vaccination (data not shown). In addition, i.n. P(O+C) caused the increase of spleen weight (FIG. 4D). We also analyzed the histology of lungs in immunized mice, and found that, compared to mice immunized by i.n. O+C, those receiving i.n. P(O+C) had more cellular infiltration but no obvious lung injuries (FIG. 3E). Taken together, this novel PLGA nanoparticle vaccine with CpG adjuvant and peptides induced robust T cell immunity with tolerable pulmonary immunopathology.

The Peripheral Prime and Local Boost Vaccination Strategy with Nanoparticle Vaccines Co-Encapsulating Peptides and CpG Enables an Optimal Protection Against IAV Infection

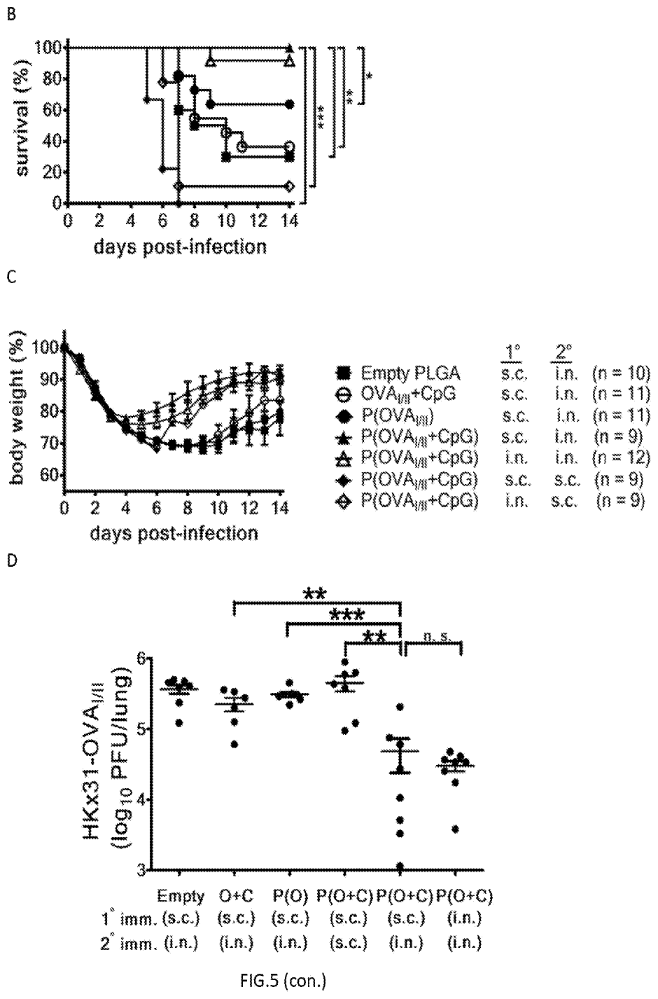

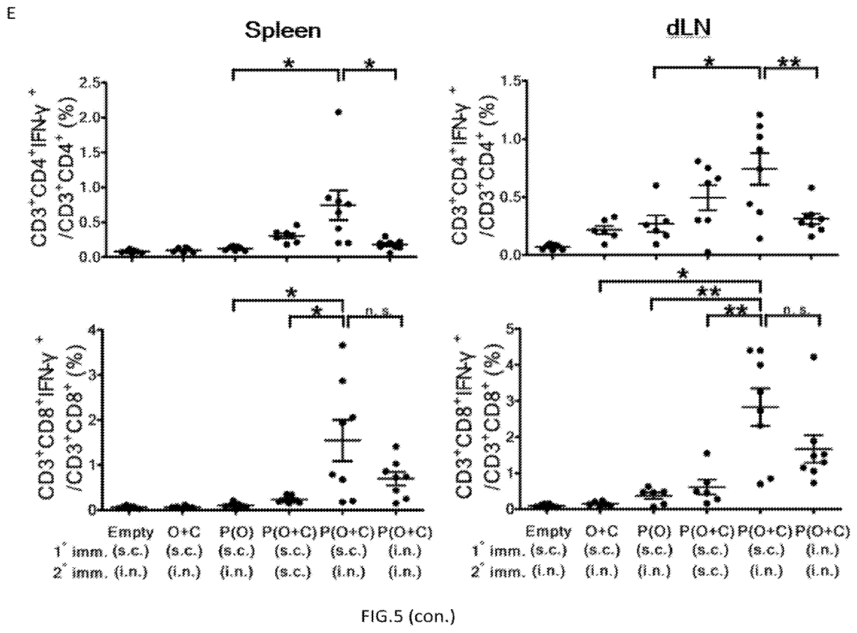

We next determined the protective efficacy of P(O+C) vaccines primed and boosted via various combinations of routes. For comparison purposes, groups P(O) and O+C mice were immunized by the peripheral (s.c.) prime and local (i.n.) boost strategy. Four weeks after boosting, mice were challenged by i.n. instillation of HKx31-OVA_{I/II} (FIG. 5A). The host protection was determined by the body weight and the survival rate of infected mice (FIGS. 5B and C). Interestingly, mice that were primed by either s.c. or i.n. P(O+C) and boosted by i.n. P(O+C) manifested the lowest body weight loss and the best survival outcome. These i.n. boosted groups of mice recovered on as early as day 5 post influenza virus challenge, while mice of the other groups either died during the infection, or did not start to recover until day 9 (FIG. 5C). The protection was especially pronounced in mice receiving s.c. prime and i.n. boost vaccination, and none of them died (FIG. 5B). In contrast, mice that were either s.c. or i.n. primed and s.c. boosted by P(O+C) were very susceptible to infection-caused deaths. In addition, P(O) and O+C groups were also s.c. primed—i.n. boosted, yet the protection was not as efficient as P(O+C). Notably, groups P(O+C) with s.c. prime/i.n. boost or i.n. prime/s.c. boost had the lowest lung viral loads, consistent with the higher protection rates of these two groups (FIG. 5D). In addition, we also demonstrated that mice with s.c. prime and i.n. boost by P(O+C) elicited the strongest CD8⁺ T cell responses in both spleens and dLNs (FIG. 5E). Mice that were i.n. primed and i.n. boosted had the second best CD8⁺ T cell responses. All other vaccine formula and immunization strategies were unable to induce effective antiviral T cell immunity by the experimental procedures, thereby the mice were left with high replicating virus titers. Collectively, the above data clearly demonstrated that the local (i.n.) boosting strategy, the PLGA nanoparticle vaccine carrier, and CpG adjuvant were critical for induction of the protective T cell immunity against IAV infection.

Nanoparticles with Authentic Peptides Targeting Conserved Influenza T Cell Epitopes Protect Against IAVs of Different Strains and Subtypes

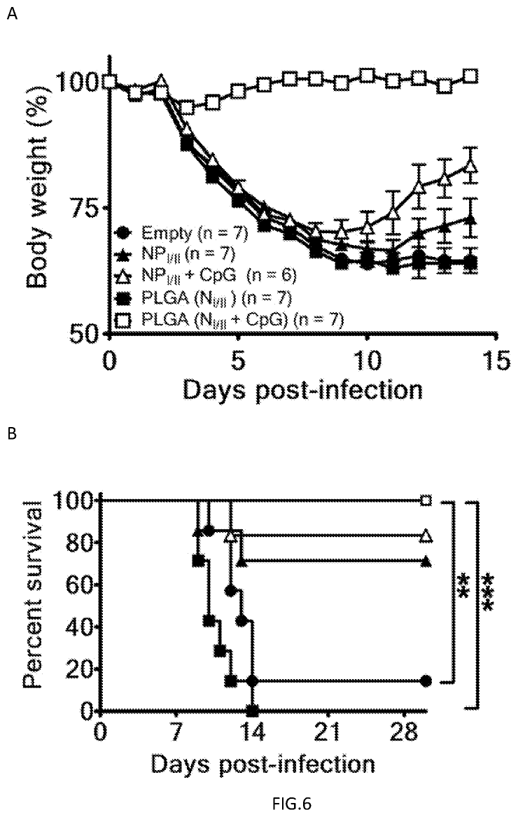

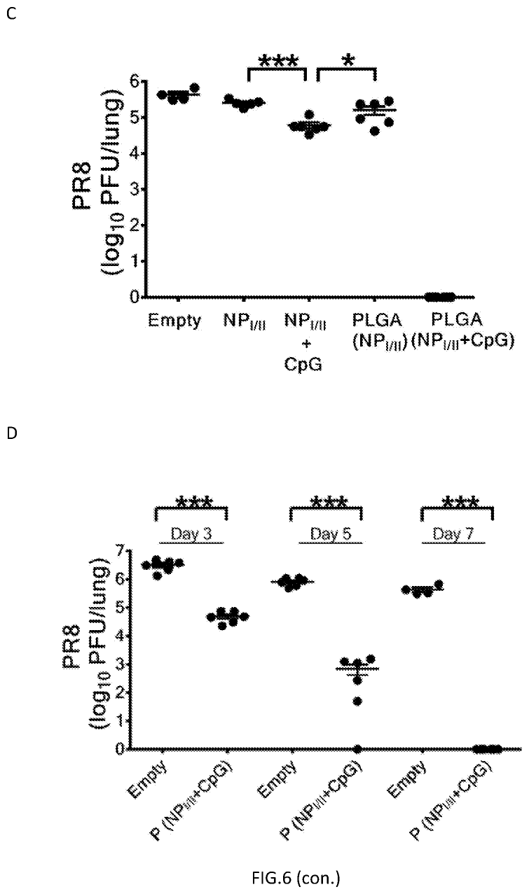

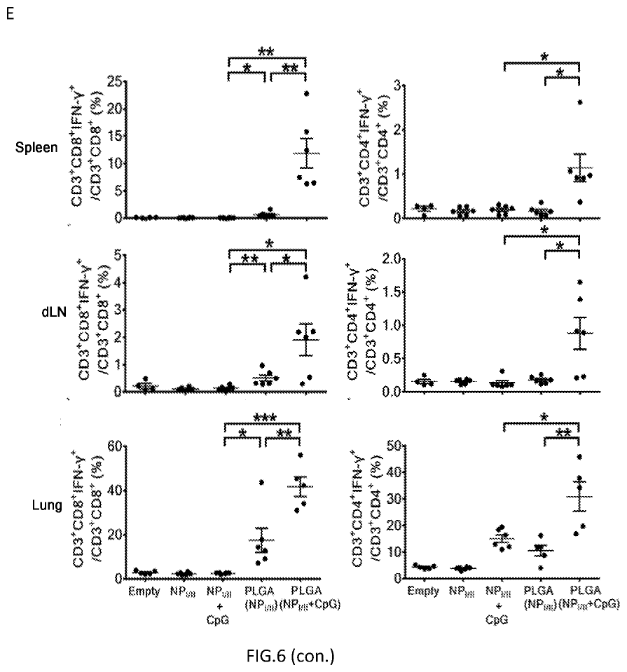

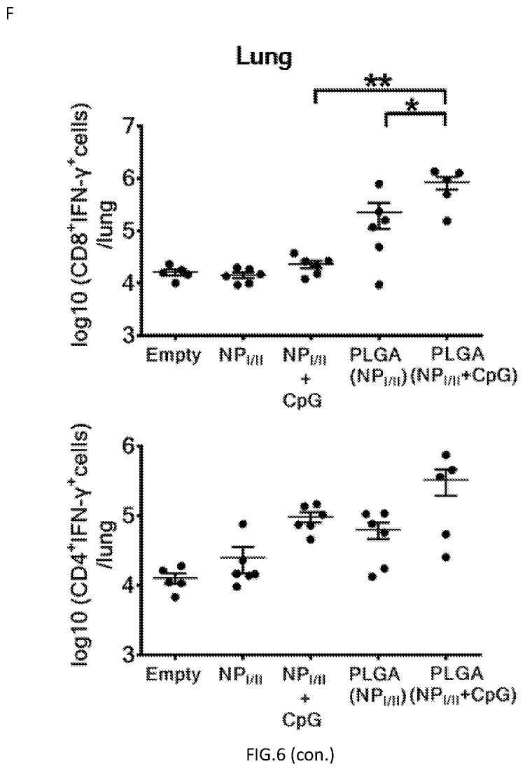

Since OVA_{I/II} peptides are not real influenza antigenic peptides, we then utilized two antigenic peptides NP₃₆₆₋₃₇₄ and NP₃₁₁₋₃₂₅ (NP_{I/II}) derived from the authentic influenza virus nucleocapsid protein (NP) of PR8 strain to validate the protectivity of our novel nanoparticle peptide vaccines against lethal IAV infection. We found that NP_{I/II} and CpG-encapsulating PLGA nanoparticle vaccines provided full protection against IAV infection when they were administered with peripheral prime (s.c.) and local (i.n.) boost strategy, and all the mice of this group survived and recovered from body weight loss much faster than all the other groups (FIGS. 6A and B). Very interestingly, only the mice immunized with s.c./i.n. P(NP_{I/II}+CpG) exhibited undetectable viral loads on day 7 post infection, but all the other groups of mice still had high viral loads (>10⁴ p.f.u per lung) (FIG. 6C). Analysis of the kinetics of the lung viral loads following lethal IAV infection revealed the rapid clearance of replicating viruses in lungs of mice receiving s.c. prime and i.n. boost P(NP_{I/II}+CpG). Their lung vial loads were significantly lower than the mice immunized by empty PLGA from day 3 post infection, and became undetectable on day 7 post infection. In contrast, the lung viral loads of mice with empty PLGA declined very slowly through day 7 post infection (FIG. 6D). We also measured the NP_{I} and NP_{II}-specific CD4 and CD8 T cell responses, and found that the mice immunized by s.c./i.n. P(NP_{I/II}+CpG) exhibited highest NP_{I/II}-specific CD4 and CD8 T cell immunity, particularly in lungs (FIG. 6E-F).

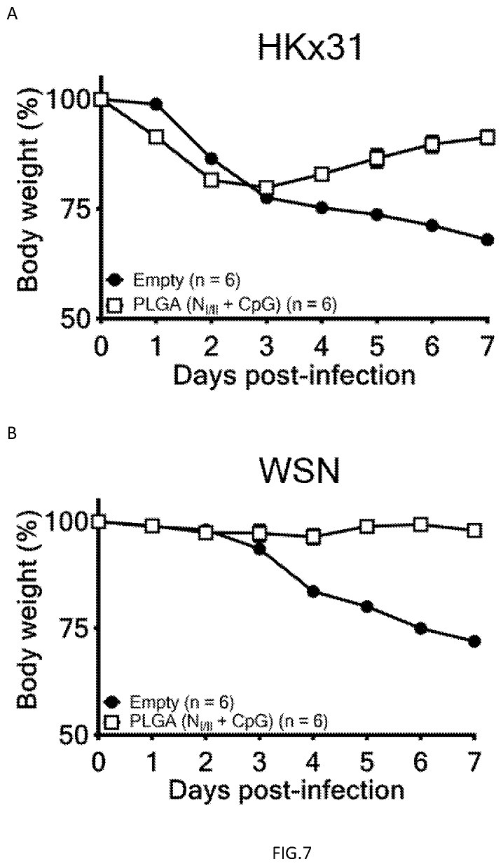

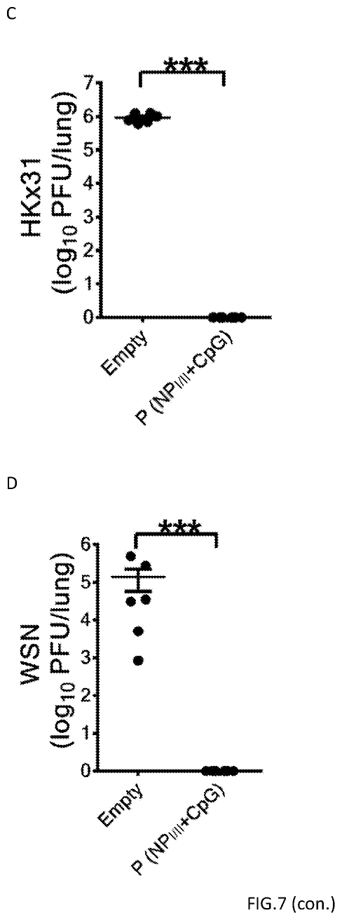

T cell vaccine is considered superior to current neutralizing antibody-stimulating vaccines for its potential to provide cross-protection against a wide spectrum of IAVs. Therefore, we further examined whether this novel nanoparticle vaccine could protect against IAVs of different strains and subtypes, namely, WSN (H1N1), and HKx31(H3N2), which share the common NP_{I/II} peptides with PR8. Our results showed that the P(NP_{I/II}+CpG) vaccine could also provide full protection against WSN and HKx31 (FIG. 7). The two groups of mice exhibited different dynamic change of body weight. The WSN-infected mice, like PR8-infected mice, did not show significant loss of body weight after infection, whereas HKx31-infected experienced an initial drop of body weight but recovered quickly (FIGS. 7A and C). Nevertheless, both WSN and HKx31-infected mice had undetectable viral loads on day 7 post infection (FIGS. 7B and D). These results indicate that our CpG-adjuvanted peptide-based nanoparticle vaccines can induce protective T cell immunity against a wide spectrum of IAVs.

### The Peripheral Prime and Local Boost Vaccination Strategy Generates Robust Resident Memory T Cells and Superior Circulatory Memory T Cells

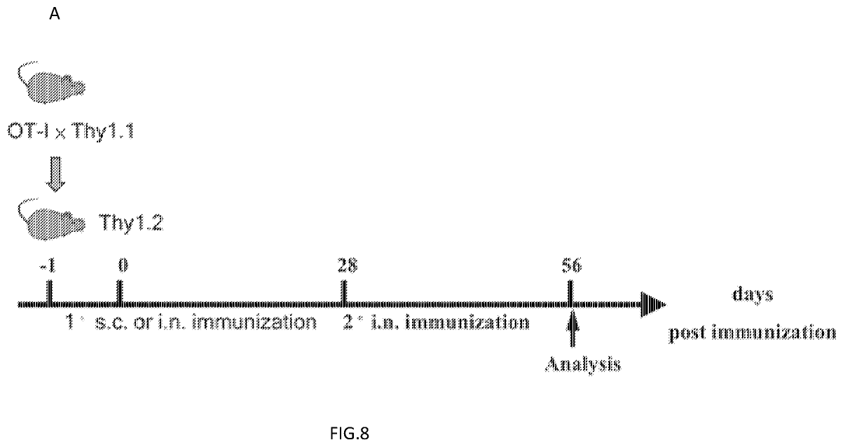

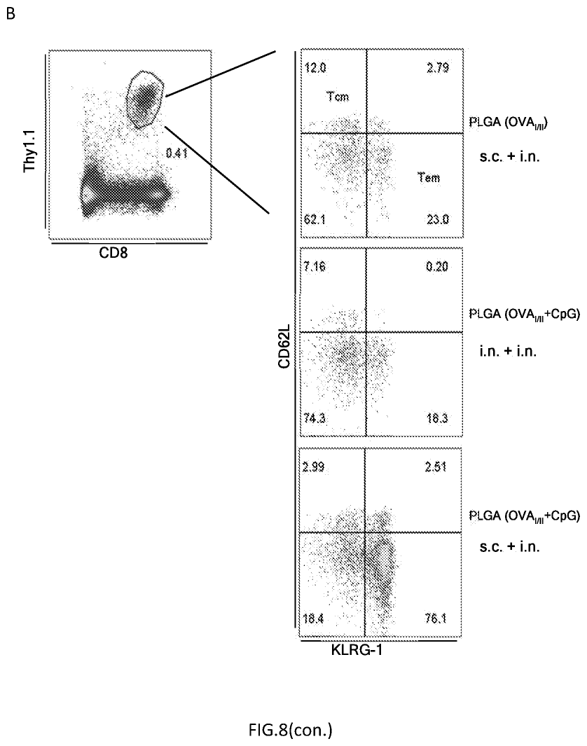

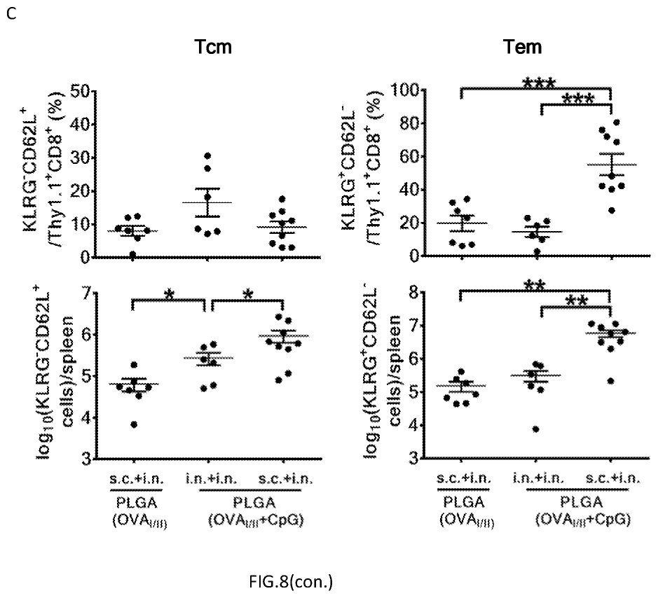

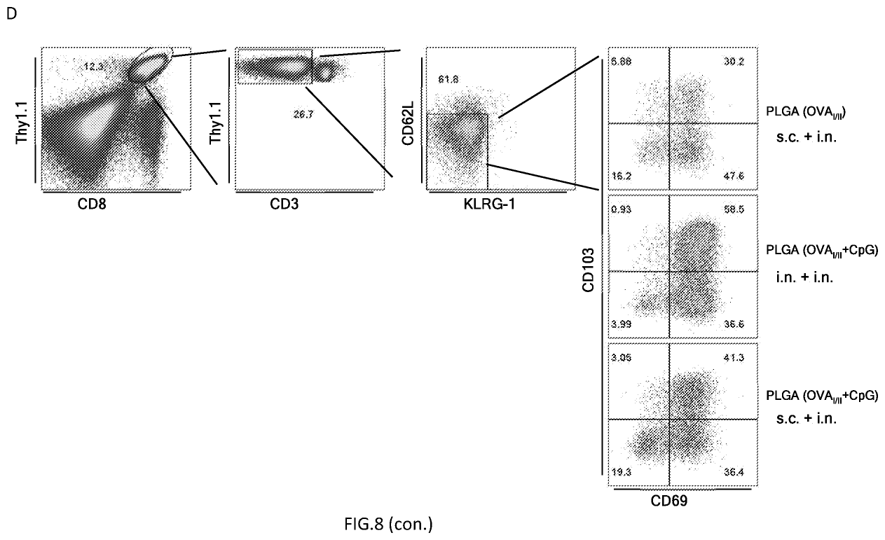

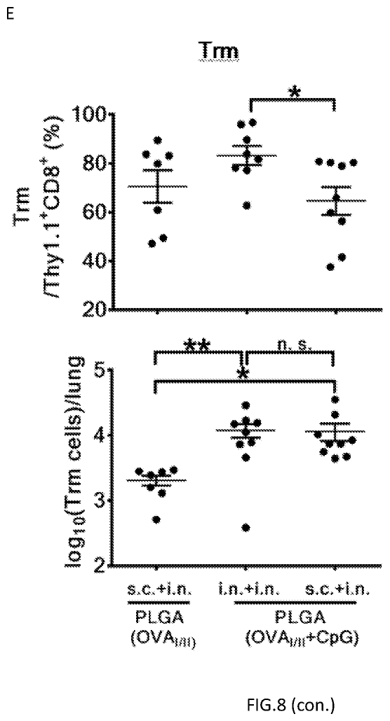

We further investigated the association between P(O+C)-derived protection and antigen-specific memory T cells by utilizing the adoptive transfer model. Naive Thy1.1⁺OT-I CD8 T cells isolated from splenocytes were adoptively transferred to WT Thy1.2⁺ C57BL/6 mice, which were subsequently immunized by s.c./i.n. P(O), i.n./i.n. P(O+C), or s.c./i.n. P(O+C). One month after the boosting, subpopulations of memory T cells, including Tcm, Tem and Trm, were analyzed by flow cytometry. Based on the expression of KLRG and CD62L, Tcm was defined as KLRG^{low}CD62^{high}, and Tem was KLRG^{high}CD62L^{low} (FIG. 8A). Our analysis showed mice that were s.c. primed and i.n. boosted with P(O+C) generated significantly more Tem and Tcm cells in spleens than mice with s.c./i.n. P(O) and mice with i.n./i.n. P(O+C) (FIG. 8B). Trm cells were determined by in vivo staining and the expression of CD69 or CD103 (FIG. 8C). The i.n./i.n. P(O+C) and s.c./i.n. P(O+C) groups had more CD69⁺ or CD103⁺ Trm cells than the s.c./i.n. P(O) group (FIG. 8D, lower panel). Although the i.n./i.n. P(O+C) group had higher percentage of CD69⁺ or CD103⁺ Trm cells than the s.c./i.n. P(O+C) group, the total number of Trm cells of these two groups were not significantly different. Collectively, compared with mice immunized by i.n./i.n. P(O+C), mice immunized by s.c./i.n. P(O+C) exhibited a similar level of lung Trm cells but significantly more circulatory memory T cells (Tcm and Tem). (FIG. 8E)

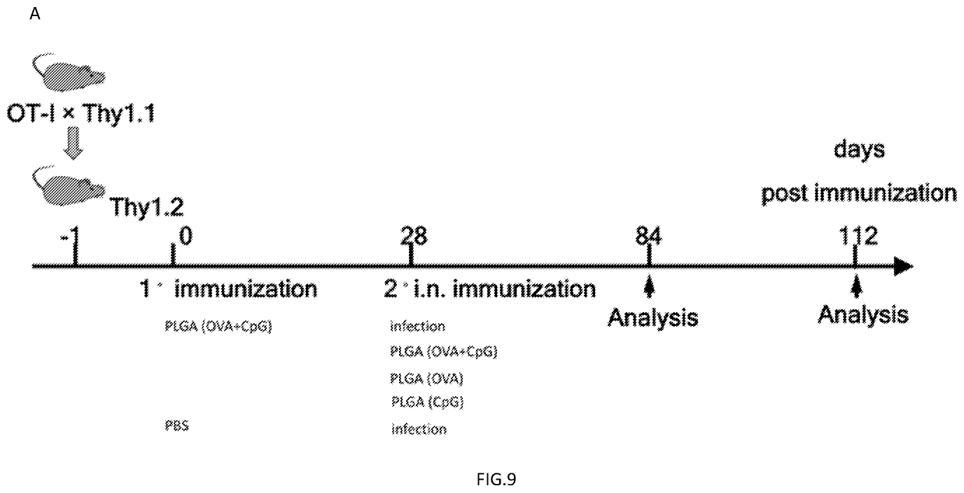

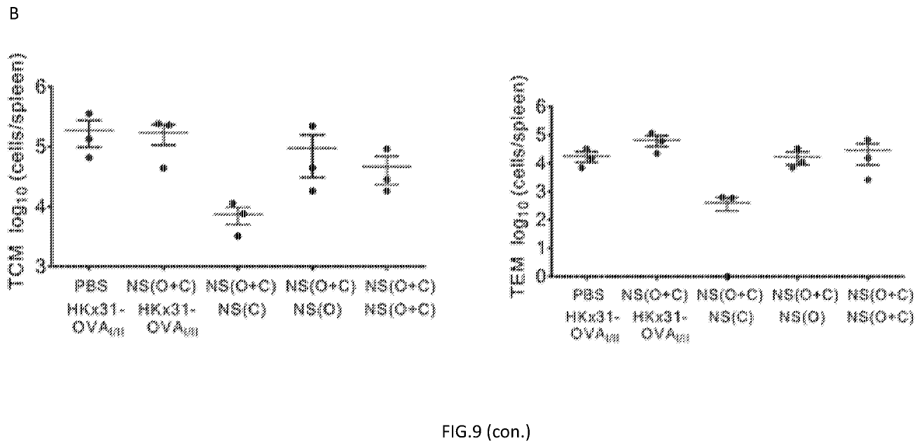

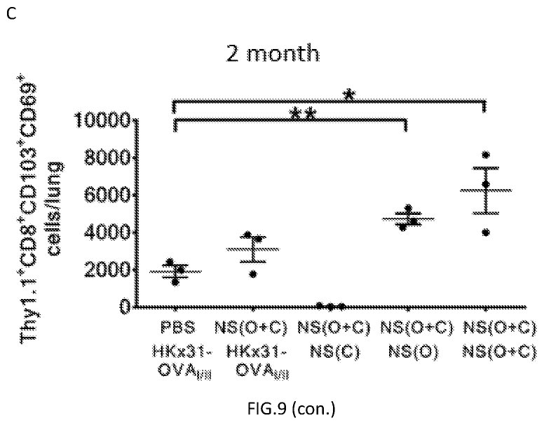

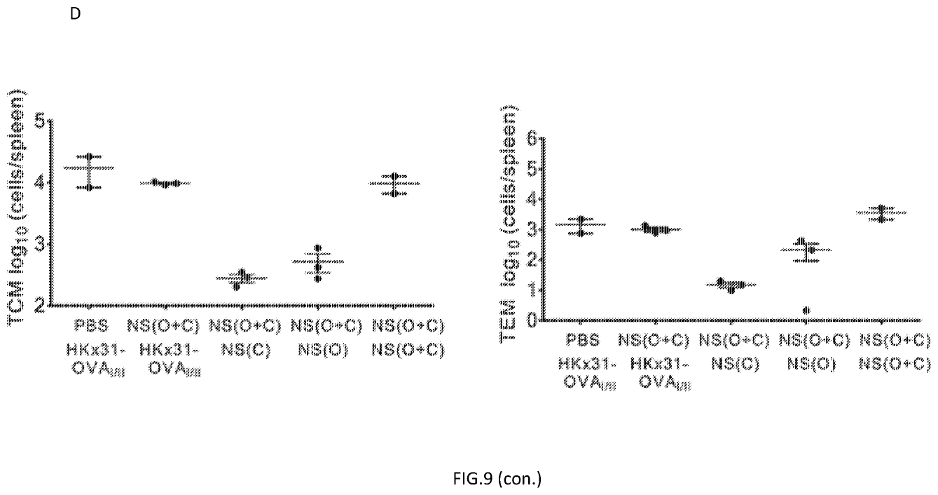

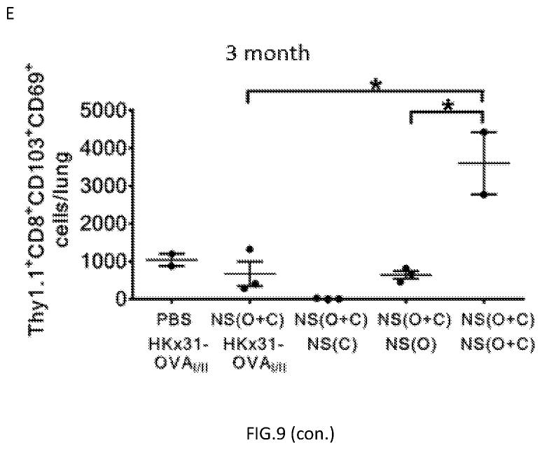

The Combinatorial Nanoparticle Vaccine with Class I and Class II HLA-Restricted Antigenic Peptides Plus CpG Elicits Durable Resident Memory T Cells
This experiment aimed to determine the durability of resident memory T cells (Trm) elicited by the combinatorial nanoshell (PLGA) vaccine. We utilized the immunization strategy with the peripheral priming and local boosting, which has been demonstrated to induce excellent circulatory and lung-resident memory T cells. To measure the antigen-specific memory T cells, naive Thy1.1+OT-I CD8+ T cells isolated from splenocytes were adoptively transferred to WT Thy1.2+ C57BL/6 mice, which were first subcutaneously (s.c.) primed with nanoshell NS(OVA_{I/II}+CpG). 28 days later, mice were intranasally (i.n.) boosted with nanoshell NS(OVA_{I/II}+CpG), or in comparison, with NS(OVA_{I/II}), NS(CpG) or i.n. infection with HKx31-OVA_{I/II} as the controls. A group of mice were PBS primed and then i.n. infected by HKx31− OVA_{I/II}, and served the infection-only control. The immunization protocol with indicated strategies are illustrated in FIG. 9A. Two months (56 days) or three months (84 days) after boosting, subpopulations of memory T cells, including Tcm, Tem, and Trm, were analyzed by flow cytometry. Our analysis showed, at 2 months or 3 months after boosting, mice that were s.c. primed and i.n. boosted with NS (OVA_{I/II}+CpG) generated similar levels of Tem and Tcm cells compared to primary or secondary influenza virus infection (FIG. 9B, D). Trm cells were determined by in vivo staining through the expression of CD69 or CD103. Interestingly, mice with s.c./i.n. NS(OVA_{I/II}+CpG) had the highest number of OT-I Trm cells, and had significantly more Trm cells than mice with either primary or boosted influenza virus infection (FIG. 9C, E). In addition, mice immunized with s.c./i.n. NS(OVA_{I/II}+CpG) also generated significantly more Trm cells than mice primed with s.c. NS(OVA_{I/II}+CpG) and boosted with i.n. NS(OVA_{I/II}) or i.n. NS(CpG), indicating the critical roles of antigen and CpG adjuvant in promoting the establishment of durable Trm cells by i.n. boosting. Collectively, our results demonstrate that the combinatorial nanoshell vaccine with appropriate antigenic peptides and strong adjuvant CpG is able to elicit durable antigen-specific Trm cells in lungs, even superior to natural influenza virus infection.

### Nanoparticle-Taking CD11c-Positive Dendritic Cells Mediates the Stimulation of T Cells in Lymph Nodes

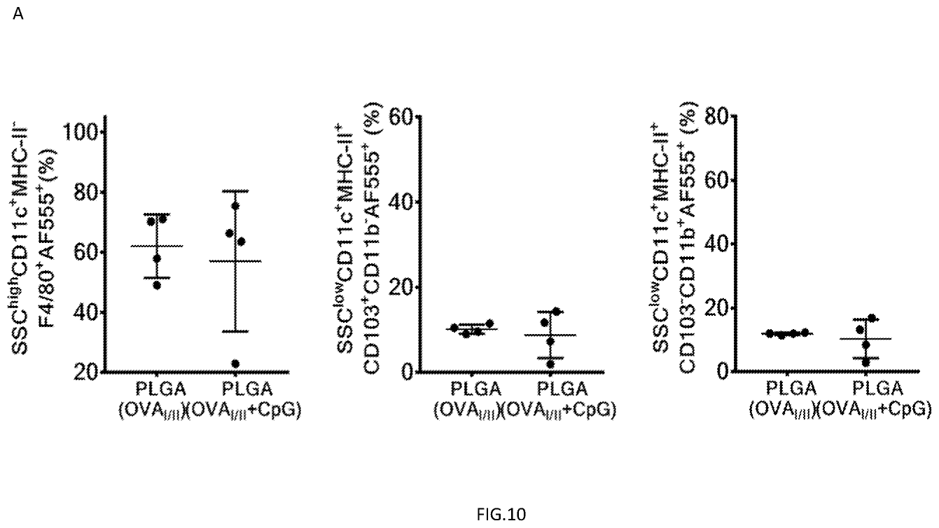

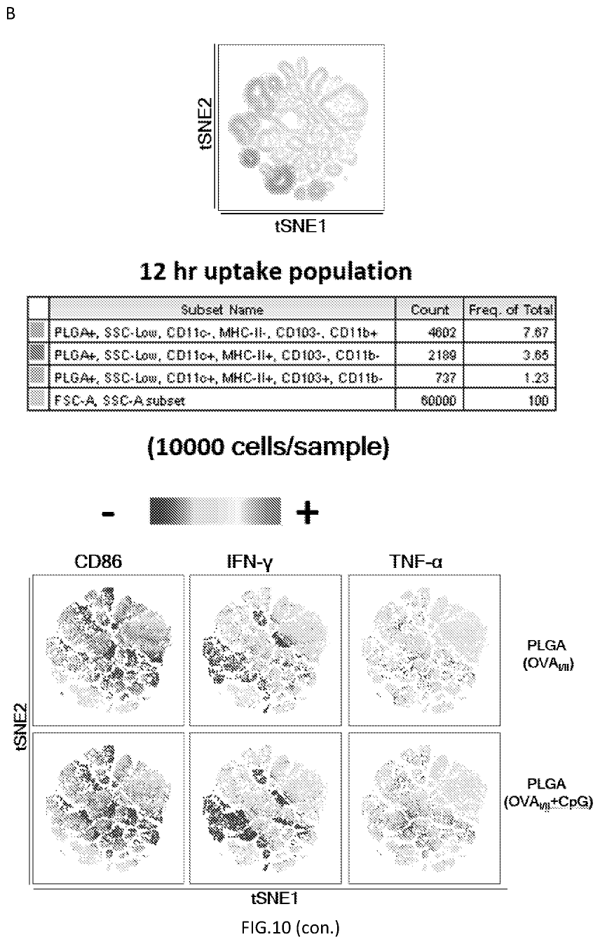

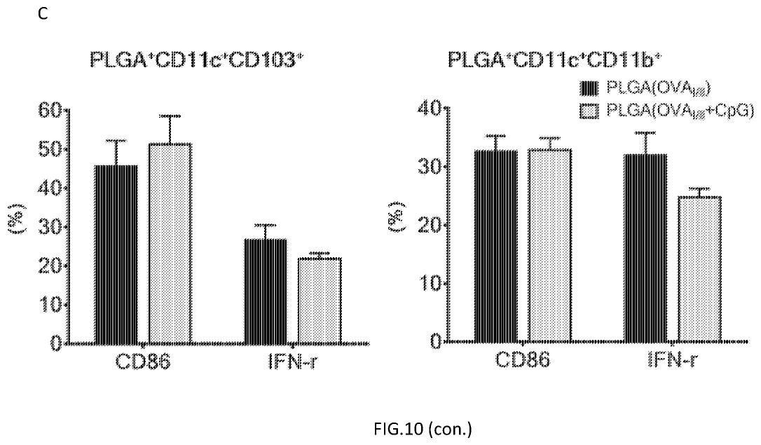

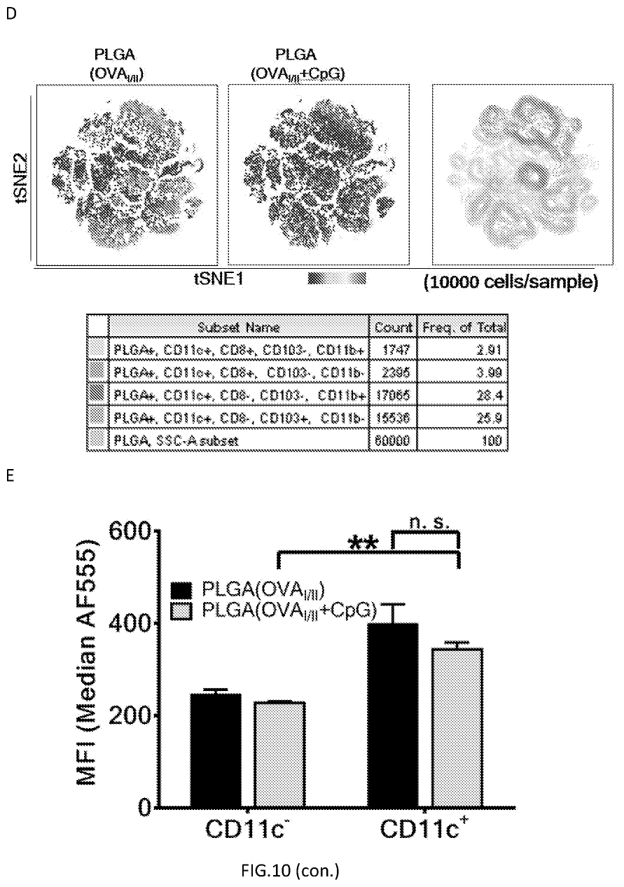

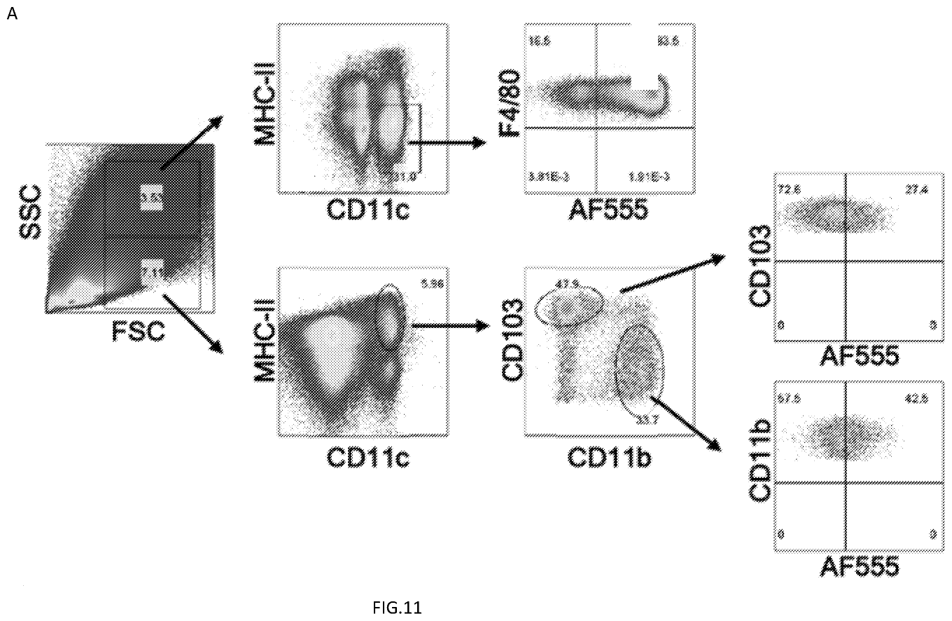

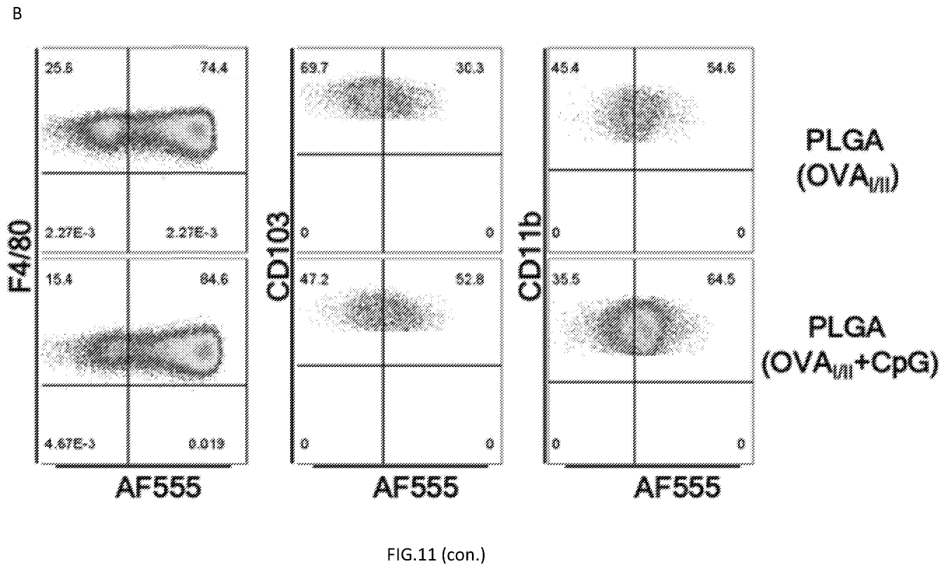

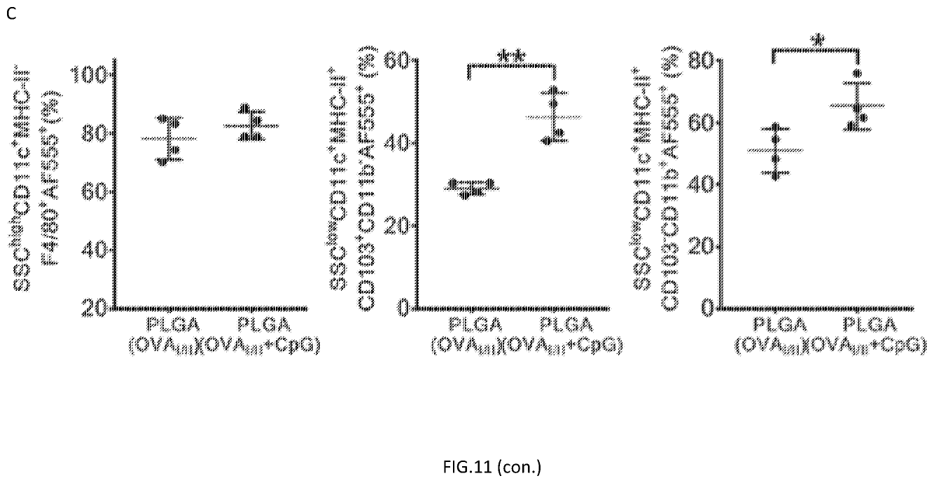

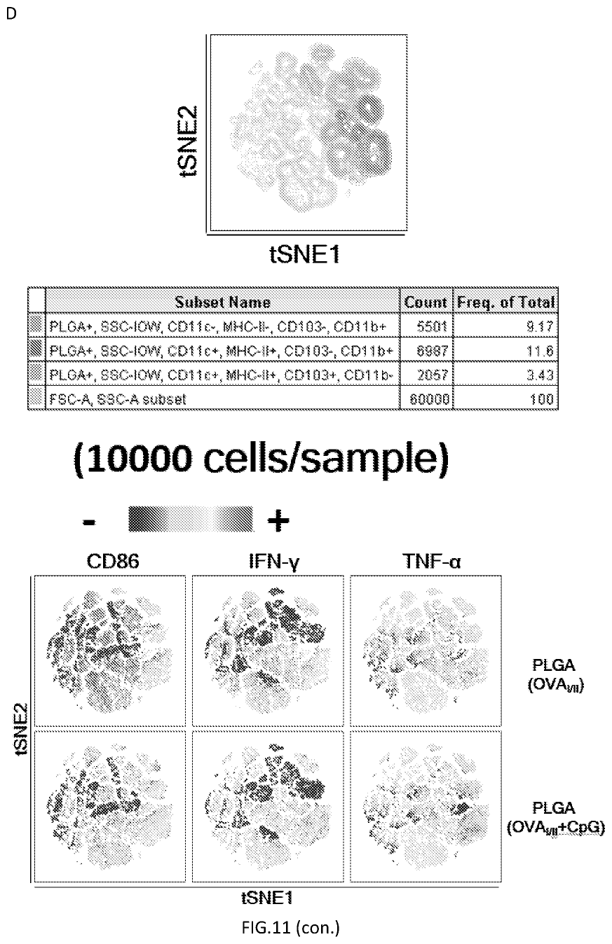

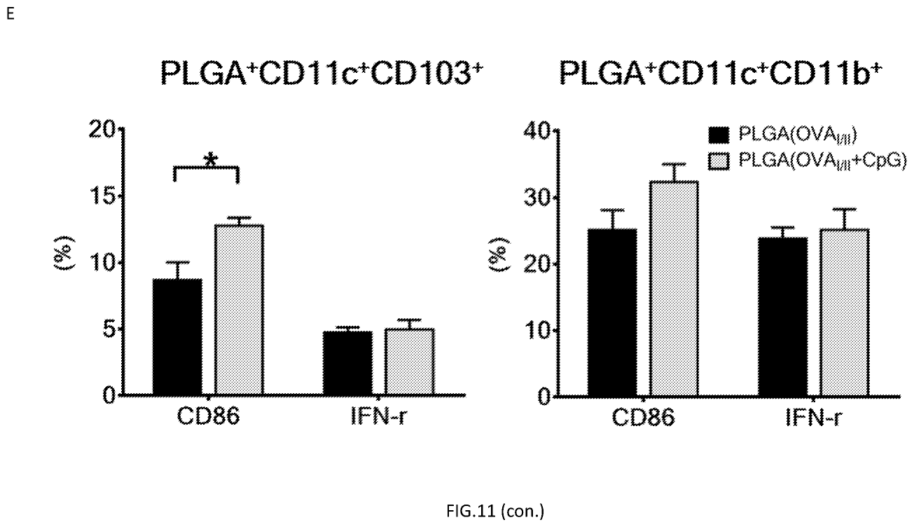

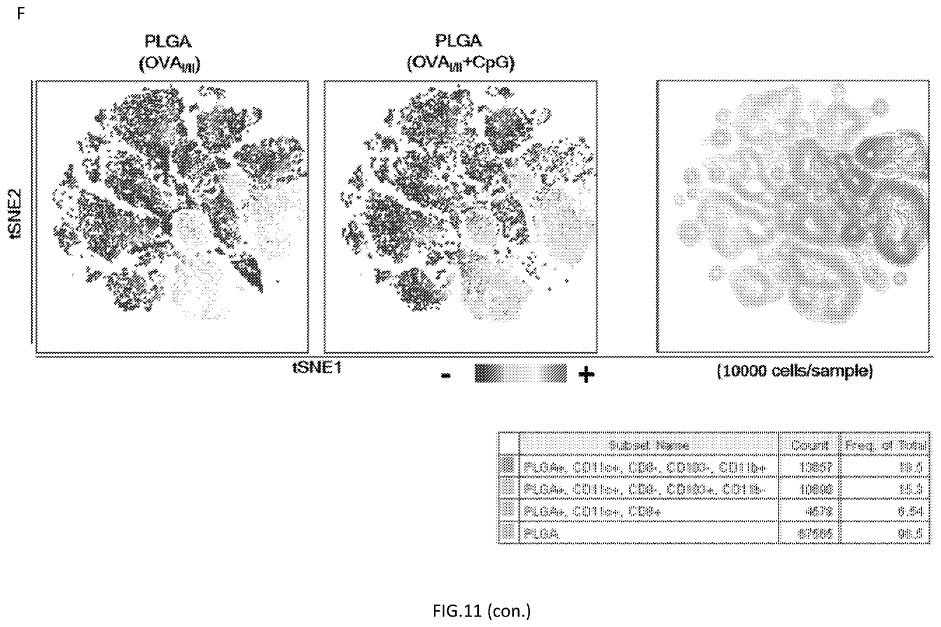

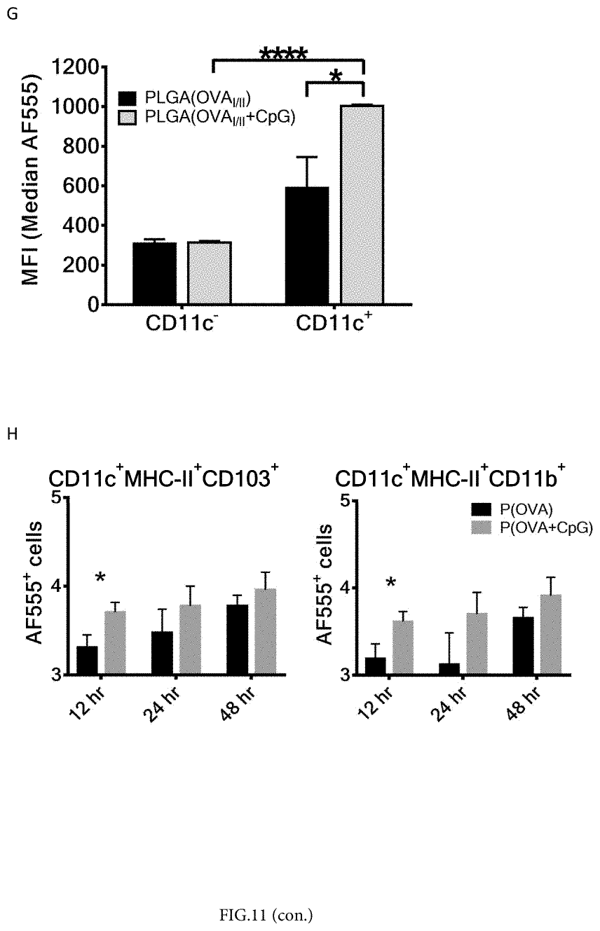

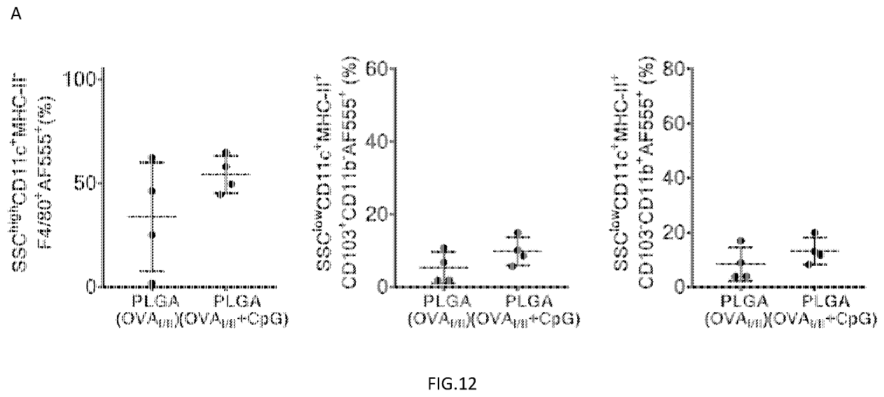

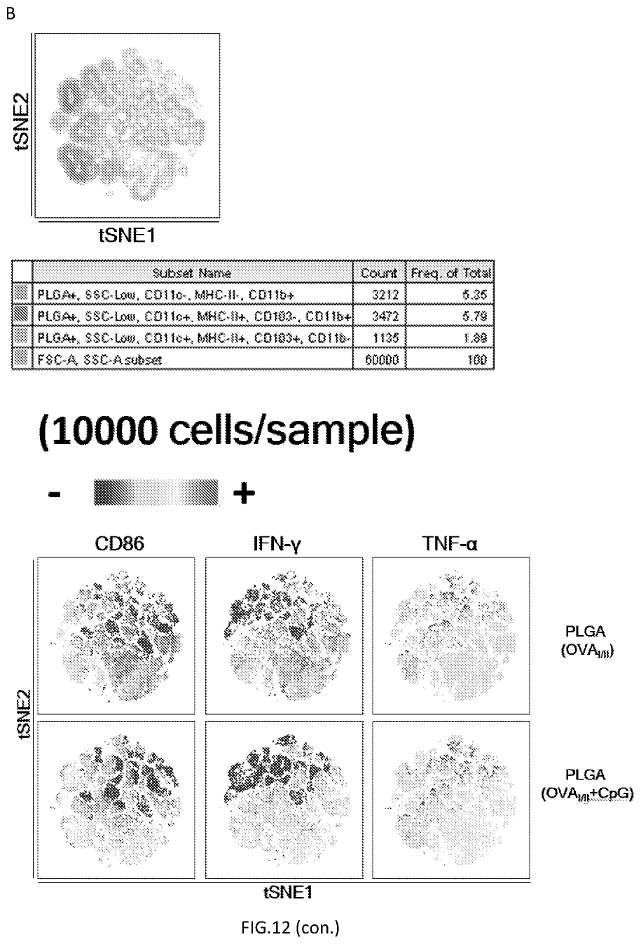

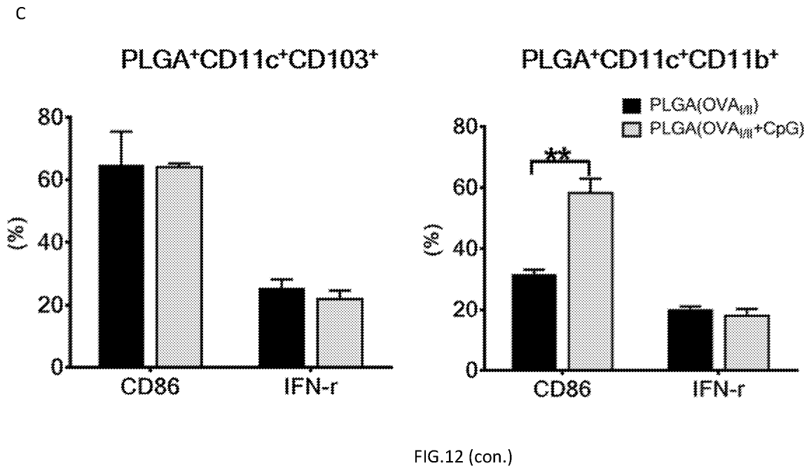

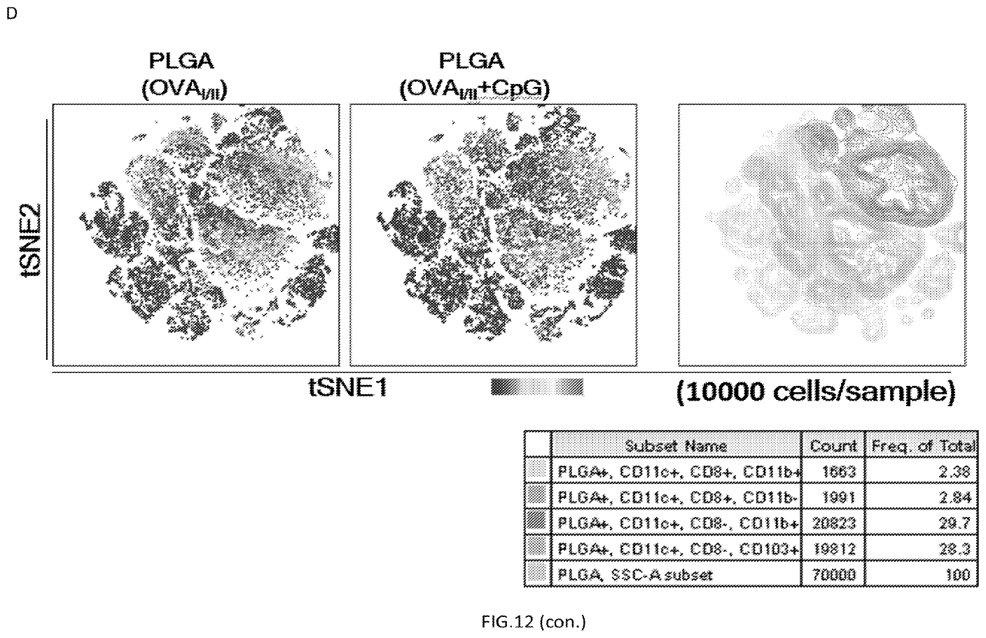

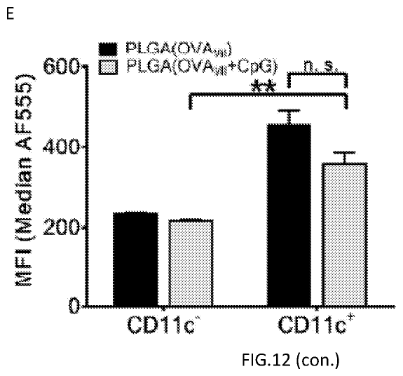

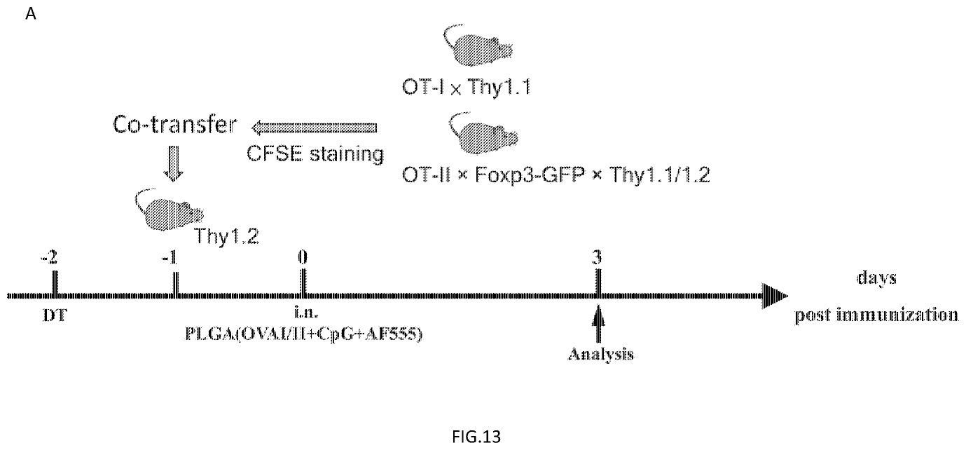

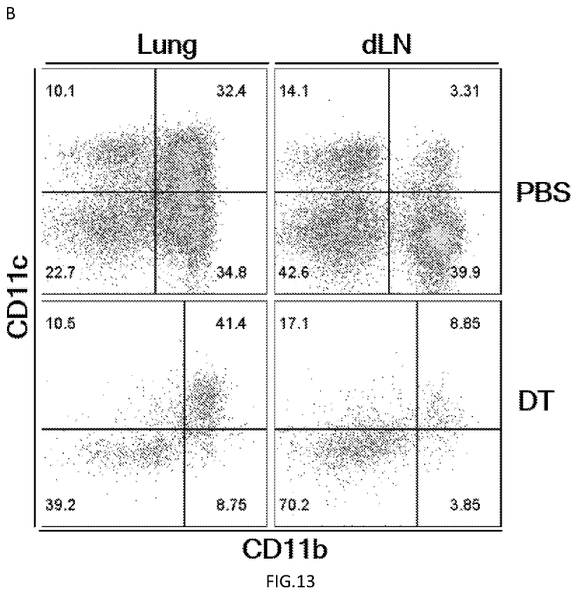

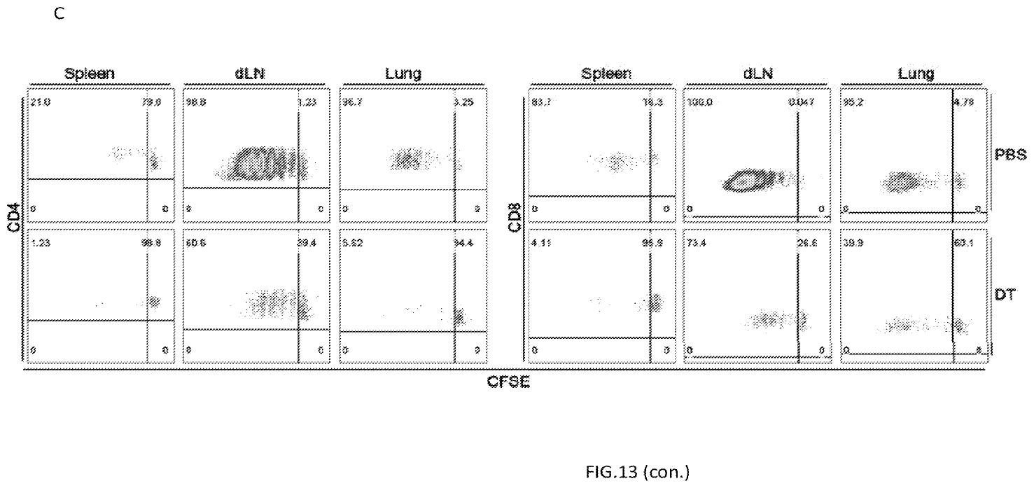

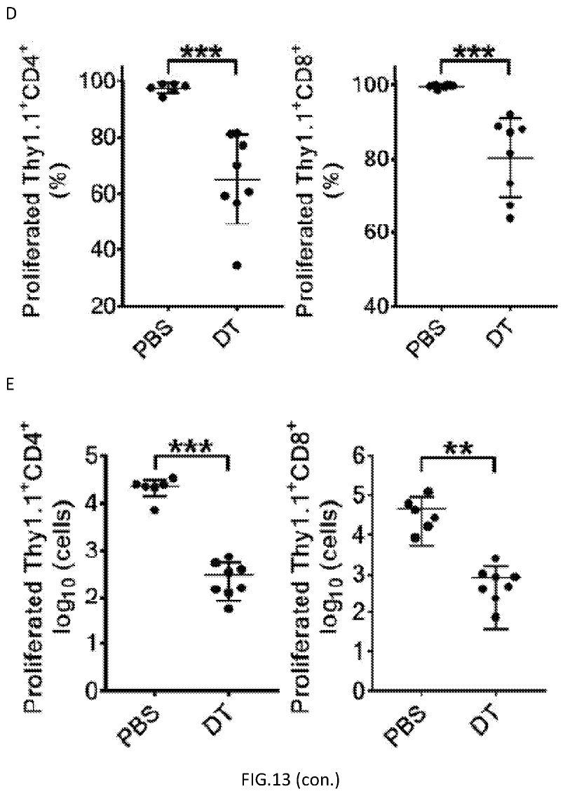

We further investigated the uptake and transport of nanoparticles in vivo. We produced nanoparticles containing tracking dye AF555 (green fluorescent). Following intranasal priming, the uptake of nanoparticles were determined by analysis of fluorescent (AF555)-taking cells isolated from lungs and dLNs at 12, 24 and 48 hours post immunization (FIG. 10, FIG. 11 and FIG. 12). We found that nanoparticles in lungs were taken by a significant portion of SSC^{high}CD11c⁺F4/80⁺ macrophages and SSC^{low}CD11c⁺ conventional DCs (cDCs), including CD103⁺CD11b⁻ and CD103⁻CD11b⁺ cDCs (FIG. 11A). Uptake of nanoparticles by macrophages and DCs peaked at 24 hours post immunization, and CpG adjuvant significantly increased the uptake of nanoparticles by CD103⁺CD11b⁻cDCs (50% vs. 30%, p<0.01) and CD103⁻CD11b⁺cDCs (70% vs. 50%, p<0.05), but not by macrophage (82% vs. 78%, p>0.05) (FIG. 11B, C). CpG adjuvant also increased the expression of CD86, a maturation marker of DCs, by CD103⁺CD11b⁻cDCs at 24 hours post immunization (FIG. 11D, E), and by CD103⁻CD11b⁺cDCs at 48 hours post immunization (FIG. 12B, C). However, CpG did not change the levels of IFN-γ and TNF-α production. In draining LNs, we found AF555⁺ nanoparticle-taking CD11c⁺ DCs, but no AF555⁺ nanoparticle-taking F4/80⁺ macrophages. In addition, nanoparticle-taking CD11c⁺ DCs in mice immunized by PLGA(OVA_{I/II}+CpG) had higher green fluorescence of AF555 than those in mice immunized by PLGA(OVA_{I/II}) (FIG. 11F-H). Collectively, the data suggest that although macrophages and DCs took nanoparticles in lungs, only CD11c⁺ DCs migrated to draining LNs. Furthermore, CpG enhanced the maturation of DCs and uptake of nanoparticles. To further determine the role of DCs in stimulating antigen-specific T cells, we utilized CD11C-DTR mice, in which CD11c⁺ APCs, primarily DCs and some macrophages, can be specifically depleted by addition of DT. Naïve Thy1.2^{+/+} CD11C-DTR mice were treated with DT for 2 consecutive days, and then adoptively transferred with CFSE-stained Thy1.1^{+/+}OT-I and Thy1.1⁺/Thy1.2⁺OT-II×Foxp3-GFP cells. Subsequently, mice were intranasally (i.n.) instilled with P(O+AF555) or P(O+C+AF555) and sacrificed for analysis 3 days later (FIG. 13A). We found that DT treatment resulted in significant reduction of CD11c⁺CD11b⁺ cells in lungs and dLNs (FIG. 13B). Depletion of CD11c-positive cells dramatically attenuated the proliferation of antigen-specific CD4 and CD8 T cells (FIG. 13C,D), and caused 2 log decrease of the cell number (FIG. 13E). Taken together, our data showed that the nanoparticles were taken by CD11c⁺ macrophages and DCs in lungs, but only nanoparticle-taking CD11c⁺ DCs migrated to draining LNs, where mature nanoparticle-taking DCs were responsible for priming vaccine-specific T cells.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### REFERENCE

1. F. Krammer et al., Influenza. Nat Rev Dis Primers 4, 3 (2018).
2. S. Sridhar, Heterosubtypic T-Cell Immunity to Influenza in Humans: Challenges for Universal T-Cell Influenza Vaccines. Front Immunol 7, 195 (2016).
3. E. B. Clemens, C. van de Sandt, S. S. Wong, L. M. Wakim, S. A. Valkenburg, Harnessing the Power of T Cells: The Promising Hope for a Universal Influenza Vaccine. Vaccines (Basel) 6, (2018).
4. P. G. Thomas, R. Keating, D. J. Hulse-Post, P. C. Doherty, Cell-mediated protection in influenza infection. Emerging infectious diseases 12, 48-54 (2006).
5. T. M. Wilkinson et al., Preexisting influenza-specific CD4+ T cells correlate with disease protection against influenza challenge in humans. Nature medicine 18, 274-280 (2012).
6. S. Sridhar et al., Cellular immune correlates of protection against symptomatic pandemic influenza. Nature medicine 19, 1305-1312 (2013).
7. Z. Wang et al., Recovery from severe H7N9 disease is associated with diverse response mechanisms dominated by CD8(+) T cells. Nature communications 6, 6833 (2015).
8. M. Koutsakos et al., Human CD8+ T cell cross-reactivity across influenza A, B and C viruses. Nature immunology, (2019).
9. S. Rosendahl Huber, J. van Beek, J. de Jonge, W. Luytjes, D. van Baarle, T cell responses to viral infections—opportunities for Peptide vaccination. Front Immunol 5, 171 (2014).
10. O. Pleguezuelos et al., A Synthetic Influenza Virus Vaccine Induces a Cellular Immune Response That Correlates with Reduction in Symptomatology and Virus Shedding in a Randomized Phase Ib Live-Virus Challenge in Humans. Clin Vaccine Immunol 22, 828-835 (2015).
11. P. H. Lin et al., Vaccine-induced antigen-specific regulatory T cells attenuate the antiviral immunity against acute influenza virus infection. Mucosal immunology, (2018).
12. R. D. de Vries, G. F. Rimmelzwaan, Viral vector-based influenza vaccines. Human vaccines & immunotherapeutics 12, 2881-2901 (2016).
13. A. Bolhassani et al., Polymeric nanoparticles: potent vectors for vaccine delivery targeting cancer and infectious diseases. Hum Vaccin Immunother 10, 321-332 (2014).
14. J. Jia et al., Interactions Between Nanoparticles and Dendritic Cells: From the Perspective of Cancer Immunotherapy. Front Oncol 8, 404 (2018).
15. F. Danhier et al., PLGA-based nanoparticles: an overview of biomedical applications. J Control Release 161, 505-522 (2012).
16. A. L. Silva, P. C. Soema, B. Slutter, F. Ossendorp, W. Jiskoot, PLGA particulate delivery systems for subunit vaccines: Linking particle properties to immunogenicity. Hum Vaccin Immunother 12, 1056-1069 (2016).
17. K. D. Zens, J. K. Chen, D. L. Farber, Vaccine-generated lung tissue-resident memory T cells provide heterosubtypic protection to influenza infection. JCI Insight 1, (2016).
18. A. Pizzolla et al., Resident memory CD8(+) T cells in the upper respiratory tract prevent pulmonary influenza virus infection. Sci Immunol 2, (2017).
19. S. N. Mueller, L. K. Mackay, Tissue-resident memory T cells: local specialists in immune defence. Nature reviews. Immunology, (2015).
20. L. Zhao et al., Nanoparticle vaccines. Vaccine 32, 327-337 (2014).
21. D. W. Mullins et al., Route of immunization with peptide-pulsed dendritic cells controls the distribution of memory and effector T cells in lymphoid tissues and determines the pattern of regional tumor control. The Journal of experimental medicine 198, 1023-1034 (2003).
22. T. Wu et al., Lung-resident memory CD8 T cells (TRM) are indispensable for optimal cross-protection against pulmonary virus infection. Journal of leukocyte biology 95, 215-224 (2014).
23. B. Slutter et al., Dynamics of influenza-induced lung-resident memory T cells underlie waning heterosubtypic immunity. Sci Immunol 2, (2017).
24. N. L. La Gruta et al., Primary CTL response magnitude in mice is determined by the extent of naive T cell recruitment and subsequent clonal expansion. The Journal of clinical investigation 120, 1885-1894 (2010).
25. S. A. Valkenburg et al., Preemptive priming readily overcomes structure-based mechanisms of virus escape. Proceedings of the National Academy of Sciences of the United States of America 110, 5570-5575 (2013).
26. L. M. Wakim, J. Smith, I. Caminschi, M. H. Lahoud, J. A. Villadangos, Antibody-targeted vaccination to lung dendritic cells generates tissue-resident memory CD8 T cells that are highly protective against influenza virus infection. Mucosal immunology 8, 1060-1071 (2015).
27. L. M. Wakim, N. Gupta, J. D. Mintern, J. A. Villadangos, Enhanced survival of lung tissue-resident memory CD8(+) T cells during infection with influenza virus due to selective expression of IFITM3. Nature immunology 14, 238-245 (2013).
28. S. Takamura et al., Specific niches for lung-resident memory CD8+ T cells at the site of tissue regeneration enable CD69-independent maintenance. The Journal of experimental medicine 213, 3057-3073 (2016).
29. C. H. GeurtsvanKessel et al., Clearance of influenza virus from the lung depends on migratory langerin+CD11b− but not plasmacytoid dendritic cells. The Journal of experimental medicine 205, 1621-1634 (2008).
30. J. T. Voeten et al., Antigenic drift in the influenza A virus (H3N2) nucleoprotein and escape from recognition by cytotoxic T lymphocytes. Journal of virology 74, 6800-6807 (2000).
31. S. A. Valkenburg et al., Acute emergence and reversion of influenza A virus quasispecies within CD8+ T cell antigenic peptides. Nature communications 4, 2663 (2013).
32. H. M. Machkovech, T. Bedford, M. A. Suchard, J. D. Bloom, Positive Selection in CD8+ T-Cell Epitopes of Influenza Virus Nucleoprotein Revealed by a Comparative Analysis of Human and Swine Viral Lineages. Journal of virology 89, 11275-11283 (2015).
33. P.-H. Lin et al., Vaccine-induced antigen-specific regulatory T cells attenuate the antiviral immunity against acute influenza virus infection. Mucosal immunology, 1 (2018).

What is claimed is:

1. A vaccine, comprising:
a polymeric hollow nanoparticle encapsulating
one or more MHC class I epitopes;
one or more MHC class II epitopes; and
an adjuvant.

2. The vaccine of claim 1, wherein the polymeric hollow nanoparticle has a diameter of 50-200 nm.

3. The vaccine of claim 1, wherein the polymeric hollow nanoparticle is substantially composed of poly(D,L-lactide-co-glycolide) (PLGA).

4. The vaccine of claim 3, wherein a lactide/glycolide ratio of the PLGA is about 40-60:60-40.

5. The vaccine of claim 1, wherein an intrinsic viscosity of the PLGA is about 0.15-0.25 dL/g.

6. The vaccine of claim 1, wherein the one or more MHC class I epitopes and the one or more MHC class II epitopes are independently antigenic peptides derived from a nucleocapsid protein of an influenza virus.

7. The vaccine of claim 6, wherein the one or more MHC class I epitopes are nucleocapsid protein₃₆₆₋₃₇₄ consisting of the amino acid sequence of SEQ ID NO: 1, and the one or more MHC class II epitopes are nucleocapsid protein₃₁₁₋₃₂₅ consisting of the amino acid sequence of SEQ ID NO: 2.

8. The vaccine of claim 1, wherein the adjuvant comprises MPLA, CpG-ODN, poly(I:C), or variants of cyclic-dinucleotides.

9. A method of manufacturing a vaccine, said vaccine comprising a polymeric hollow nanoparticle encapsulating one or more MHC class I epitopes, one or more MHC class II epitopes, and an adjuvant, comprising:
emulsifying an first solution comprising one or more MHC class I epitopes, one or more MHC class II epitopes and an adjuvant in a solvent comprising poly(D,L-lactide-co-glycolide) (PLGA);
sonicating the emulsion; and
purifying the polymeric hollow nanoparticle in the emulsion.

10. The method of claim 9, further comprising
adding a second solution to the emulsion after the sonicating step;
pouring the emulsion to water after the adding step; and
evaporating the solvent from the emulsion.

11. The method of claim 10, wherein the first solution comprises sodium bicarbonate.

12. The method of claim 11, wherein the concentration of the sodium bicarbonate ranges from 100-300 mM.

13. The method of claim 9, wherein the solvent comprises dichloromethane.

14. The method of claim 9, wherein the one or more MHC class I epitopes and the one or more MHC class II epitopes are independently antigenic peptides derived from a nucleocapsid protein of an influenza virus.

15. The method of claim 14, wherein the one or more MHC class I epitopes are nucleocapsid protein₃₆₆₋₃₇₄ consisting of the amino acid sequence of SEQ ID NO: 1, and the one or more MHC class II epitopes are nucleocapsid protein₃₁₁₋₃₂₅ consisting of the amino acid sequence of SEQ ID NO: 2.

16. The method of claim 9, wherein the adjuvant comprises MPLA, CpG-ODN, poly(I:C), or variants of cyclic-dinucleotides.

17. The method of claim 9, wherein a lactide/glycolide ratio of the PLGA is about 40-60:60-40.

18. A method of neutralizing virus infection, comprising:
priming a subject in need thereof with an vaccine, wherein said vaccine comprises a polymeric hollow nanoparticle encapsulating one or more MHC class I epitopes; one or more MHC class II epitopes and an adjuvant.

19. The method of claim 18, wherein the polymeric hollow nanoparticle is substantially composed of poly(D,L-lactide-co-glycolide) (PLGA).

20. The method of claim 19, wherein a lactide/glycolide ratio of the PLGA is about 40-60:60-40.

21. The method of claim 18, wherein an intrinsic viscosity of the PLGA is about 0.15-0.25 dL/g.

22. The method of claim 18, wherein the one or more MHC class I epitopes and the one or more MHC class II epitopes are independently antigenic peptides derived from a nucleocapsid protein of an influenza virus.

23. The method of claim 22, wherein the one or more MHC class I epitopes are nucleocapsid protein₃₆₆₋₃₇₄ consisting of the amino acid sequence of SEQ ID NO: 1, and the one or more MHC class II epitopes are nucleocapsid protein₃₁₁₋₃₂₅ consisting of the amino acid sequence of SEQ ID NO: 2.

24. The method of claim 18, wherein the adjuvant comprises MPLA, CpG-ODN, poly(I:C), or variants of cyclic-dinucleotides.

25. The method of claim 18, further comprising
boosting the subject with the vaccine.

26. The method of claim 25, wherein the priming step and the boosting step is by at least one mode selected from the group consisting of parenteral, subcutaneous, intramuscular, intravenous, intra-articular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, and transdermal.

27. The method of claim 25, wherein the priming step and the boosting step are by subcutaneous or intranasal.


SEQUENCE LISTING (2 sequences)

SEQ ID NO: 1 - protein, 9 aa, Influenza A virus
ASNENMETM

SEQ ID NO: 2 - protein, 15 aa, Influenza A virus
QVYSLIRPNENPAHK